(19) United States
(12) Patent Application Publication
(10) Pub. No.: US 2017/0248596 A1
(43) Pub. Date: Aug. 31, 2017

(54) METHODS AND COMPOSITIONS FOR OBTAINING A TUBERCULOSIS ASSESSMENT IN A SUBJECT

(71) Applicants: Becton, Dickinson and Company, Franklin Lakes, NJ (US); STELLENBOSCH UNIVERSITY, Stellenbosch (ZA)

(72) Inventors: Farida BOUZAHZAH, Cary, NC (US); Gerhard WALZL, Stellenbosch (ZA); Alex LASTOVICH, Raleigh, NC (US)

(21) Appl. No.: 15/507,222

(22) PCT Filed: Aug. 24, 2015
(86) PCT No.: PCT/US2015/046570

§ 371 Date: Feb. 27, 2017

Related U.S. Application Data
(60) Provisional application No. 62/154,996, filed on Apr. 30, 2015.
(60) Provisional application No. 62/115,958, filed on Feb. 13, 2015.
(60) Provisional application No. 62/085,032, filed on Nov. 26, 2014.
(60) Provisional application No. 62/044,045, filed on Aug. 29, 2014.

(51) Int. Cl. G01N 33/569; G01N 15/14

(52) U.S. Cl. CPC G01N 33/5695; G01N 15/1459; G01N 2333/70596; G01N 2800/26; G01N 2800/52; G01N 2015/1006

(57) ABSTRACT

Methods for obtaining a tuberculosis assessment in a subject are provided. Aspects of the methods include identifying a subpopulation of a cellular sample of the subject having an expression level for a tuberculosis host biomarker below a threshold expression level to produce a biomarker signature; and obtaining a tuberculosis assessment for the subject from the biomarker signature. Aspects of the invention further include reagents, devices, systems, and kits thereof that find use in practicing the subject methods are provided. The methods and compositions find use in a variety of applications, including diagnosis and monitoring of TB.

### CROSS-REFERENCE TO RELATED APPLICATIONS

Pursuant to 35 U.S.C. §119 (e), this application claims priority to the filing dates of U.S. Provisional Patent Application Ser. No. 62/154,996 filed Apr. 30, 2015; U.S. Provisional Application No. 62/115,958 filed on Feb. 13, 2015; U.S. Provisional Application No. 62/085,032 filed on Nov. 26, 2014 and U.S. Provisional Application No. 62/044,045 filed on Aug. 29, 2014; the disclosures of which applications are incorporated herein by reference.

### INTRODUCTION

TB disease is caused by a bacterium called Mycobacterium tuberculosis. The bacteria commonly infect the lungs, but TB bacteria can also infect any other part of the body, including, e.g., the kidney, the spine, and the brain. If not treated properly, TB disease can be fatal. TB is generally transmitted through the air from an infected person to a second person, e.g., when a person with a TB infection or TB disease of the lungs or throat coughs, sneezes, speaks, or sings and airborne bacteria are inhaled by a second person.

About one-third of the world's population has latent TB, which means people have been infected by TB bacteria but are asymptomatic and cannot transmit the disease.

According to the World Health Organization (WHO), TB is second only to HIV/AIDS as the greatest killer worldwide due to a single infectious agent. For example, in 2012, 8.6 million people fell ill with TB and 1.3 million died from TB. Furthermore, TB is highly prevalent in the developing world with over 95% of TB deaths occurring in low- and middle-income countries. TB is among the top three causes of death for women aged 15 to 44. TB is also highly prevalent in children. For example, in 2012, an estimated 530,000 children became ill with TB and 74,000 HIV-negative children died of TB. Co-infection of TB and HIV remains a significant health burden as TB is a leading killer of people living with HIV causing one fifth of all deaths. Multi-drug resistant TB is present in virtually all countries surveyed by the WHO.

### SUMMARY

Methods for obtaining a tuberculosis assessment in a subject are provided. Aspects of the methods include identifying a subpopulation of a cellular sample of the subject having an expression level for a tuberculosis host biomarker below a threshold expression level to produce a biomarker signature; and obtaining a tuberculosis assessment for the subject from the biomarker signature. Aspects of the invention further include reagents, devices, systems, and kits thereof that find use in practicing the subject methods are provided. The methods and compositions find use in a variety of applications, including diagnosis and monitoring of TB.

### BRIEF DESCRIPTION OF THE FIGURES

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1 provides Table 1 which shows the expression of a selection of biomarkers measured before and during TB therapy.

FIG. 2 provides density curves illustrating the distribution of the expression of CD126 measured before and during TB therapy.

FIG. 3 provides density curves illustrating the distribution of the expression of CD62L measured before and during TB therapy.

FIG. 4 provides density curves illustrating the distribution of the expression of CD126 measured in various patient groups.

FIG. 5 provides density curves illustrating the distribution of the expression of CD62L measured in various patient groups.

FIG. 6 provides the expression of CD120b, CD126, and CD62L marker levels in various patient groups and times before and during TB therapy with associated significance values.

FIG. 7 provides the individual patient data pertaining to CD126 marker levels at various times before and during TB therapy.

FIGS. 8A to 8C provide Flow-cytometry plots pertaining to the co-expression of CD4 and CD126 on an exemplary patient's cells at various times before and during TB therapy.

FIG. 9 provides Table 2 which provides the results of paired t-test analysis for a selection of biomarkers at various times before and during TB therapy.

FIG. 10 provides Table 3 which provides the results of independent two-sample t-test analysis for a selection of biomarkers in various patient groups.

FIG. 11 provides the expression of CD19, CD3, CD4, CD4.1, CD56, CD57 and CD8 marker levels in various patient groups and times before and during TB therapy with associated significance values.

FIG. 12 provides the expression of CCR7, CD127, CD27, CD28, and HLA-DR marker levels in various patient groups and times before and during TB therapy with associated significance values.

### DETAILED DESCRIPTION

Methods for obtaining a tuberculosis assessment in a subject are provided. Aspects of the methods include identifying a subpopulation of a cellular sample of the subject having an expression level for a tuberculosis host biomarker below a threshold expression level to produce a biomarker signature; and obtaining a tuberculosis assessment for the subject from the biomarker signature. Aspects of the invention further include reagents, devices, systems, and kits thereof that find use in practicing the subject methods are provided. The methods and compositions find use in a variety of applications, including diagnosis and monitoring of TB.

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Methods

The present disclosure provides methods for making TB assessments of subjects. By making a “TB assessment” or a “TB assessment of a subject”, it is meant an evaluation of a subject with respect to TB, which evaluation may be in a variety of formats, including but not limited to diagnosing the presence of TB in the subject, clinically monitoring TB in a subject (e.g., the progression of TB in the subject), etc. Diagnosing TB includes, e.g., diagnosing TB disease and, in some instances, discriminating, e.g., between latent TB infection, progressive TB disease, drug resistant TB disease, etc. An assessment that includes a TB diagnosis may also include a determination of a treatment or a course of treatment for a patient suspected of having a TB infection or TB disease. Clinically monitoring TB includes, e.g., evaluating the clinical progression of TB in a subject, including, e.g., evaluating treatment effectiveness and patient response to treatment, evaluating treatment endpoints, post-treatment follow-up, etc.

TB assessments may be obtained through the production and analysis of a biomarker signature. In some instances a TB assessment, e.g., a TB diagnosis, is obtained through the detection of a particular biomarker signature obtained for a subject, e.g., a subject suspected of having TB. In other instances, a TB assessment, e.g., a clinical assessment, including but not limited to, e.g., a clinical assessment of TB disease state, a clinical assessment of TB disease progression, a clinical assessment of TB treatment progression or a clinical assessment of TB treatment outcome, is obtained through the detection of a particular biomarker signature obtained for a subject known to have TB.

In certain embodiments, a TB assessment that includes the production or analysis of a biomarker signature also includes further assessments or tests, such that production or analysis of the biomarker signature is one component of a more extension evaluation protocol, which extensive evaluation protocol may include, e.g., a plurality of tests. In some instances, a TB assessment may include the production or analysis of a biomarker signature prior to, concurrent with, or following one or more additional clinical evaluations or tests. For example, a TB assessment that includes the production or analysis of a biomarker signature may be performed following a conventional clinical evaluation, including but not limited to, e.g., a conventional physical examination, conventional blood work, a conventional lung function test, a conventional TB test, etc. In other embodiments, a TB assessment consists essentially of the production or analysis of one or more biomarker signatures.

Additional clinical evaluations or tests, also referred to herein as “other clinical tests” that may contribute to a TB assessment may vary and include those tests known to be useful in assessing TB infection, TB disease, other lung diseases (e.g., those described herein), or general lung function. For example, in some instances an assessment includes one or more lung function tests, including but not limited to: Forced Vital Capacity (FVC), FVC % p, Forced Expiratory Volume in 1 Second (FEV1), FEV1% (FEV1/FVC), Peak Expiratory Flow (PEF), Forced Expiratory Flow 25-75% or 25-50% (FEF 25-75% or 25-50%), Forced Inspiratory Flow 25%-75% or 25%-50% (FIF 25-75% or 25-50%), Forced Expiratory Time (FET), Tidal Volume (TV), Maximum Voluntary Ventilation (MW), Functional residual capacity (FRC), The lung carbon monoxide diffusing capacity (DLCO), and the like. Other clinical tests that may be used as part of or in combination with a TB assessment as described herein include but are not limited to computerized tomography (CT) scan, positron emission tomography (PET) scan, combined PET-CT scan, magnetic resonance imaging (MRI), sputum smear, culture, genetic testing, proteomic testing, etc. For example, in some instances, a TB assessment may include one or more CT, PET, combined PET/CT or MRI scans and analysis of such scans, e.g., as described in Skoura et al., Int J Infect Dis. 2015, 32:87-93; Vorster et al., Mol Imaging Radionucl Ther. 2015, 5; 24(1):42; Coleman et al., Sci Transl Med. 2014, 6(265):265ra167; Chen et al., Sci Transl Med. 2014 Dec. 3; 6(265):265ra166 and Vorster et al., Curr Opin Pulm Med. 2014, 20(3):287-93, the disclosures of which are incorporated herein by reference in their entirety.

In certain embodiments, in making a TB assessment, one or more conventional TB tests may be performed in addition to the TB assessments described herein, e.g., to confirm or disconfirm a result of a previous conventional TB test. Conventional TB tests include, e.g., TB screening tests. In certain instances, conventional TB tests may be used as a component of a comprehensive TB assessment, e.g., used in conjunction with an assessment that includes a determination of a biomarker signature as described herein, which may lead to an evaluation of TB treatment or a TB diagnosis. Conventional TB tests may be used to detect latent TB and TB disease. Any convenient TB testing method may be employed, including but not limited to, e.g., the TB skin test (TST), TB blood tests, etc. Conventional TB tests are generally given by a health care provider or local health department and may be considered as screening tests. Positive reactions to conventional TB tests generally indicate a need for further tests, e.g., to confirm TB infection or to determine whether the subject has TB disease. In some instances, further testing indicated by a positive reaction to a conventional TB test is performed using the TB assessments described herein.

In certain embodiments, a TB assessment may include a determination, assessment, or measurement of biomarkers in addition to those described herein as “TB host biomarkers”. Such additional biomarkers include those biomarkers in addition to “TB host biomarkers” described herein that may be used to diagnose TB, assess TB disease state, monitor TB disease progression, evaluate TB treatment efficacy, or assess general health.

In some instances, TB assessments that include clinical monitoring, including, e.g., end of treatment assessments and follow-up assessments, may be performed to detect the occurrence of drug-resistant or multi-drug resistant TB infection, e.g., to indicate the necessity of initiation of a drug-resistant TB treatment regimen. Drug-resistant TB is caused by TB bacteria that are resistant to at least one first-line anti-TB drug. Multidrug-resistant TB (MDR TB) is resistant to more than one anti-TB drug including e.g., at least isoniazid (INH) and rifampin (RIF). In some instances, confirmation of drug-resistant and multi-drug resistant TB is performed by drug-susceptibility testing. In some instances, TB assessments that include monitoring of TB treatment, e.g., using the TB assessments described herein, may be used to detect drug-resistant and multi-drug resistant TB before or concurrent with drug-susceptibility testing.

TB assessments, as described herein, are made, either alone or in combination with other evaluations or factors, based on a biomarker signature. By “biomarker signature” is meant the presence, absence, or relative level, e.g., expression level, of one or more biomarkers as described herein. The number of biomarkers that make up a biomarker signature will vary and in some instances may range from 1 to 200, including, e.g., 1, 2, 3, 4, 5, 6, 7, 8, 9, 10, 11, 12, 13, 14, 15, 16, 17, 18, 19, 20, from 1 to 3, from 2 to 5, from 1 to 5, from 5 to 10, from 2 to 8, from 1 to 10, from 5 to 15, from 10 to 20, from 20 to 40, from 30 to 50, from 40 to 60, from 50 to 70, from 60 to 80, from 70 to 90, from 80 to 100, from 50 to 150, from 100 to 200, from 150 to 200, etc. In certain instances, a biomarker signature includes a qualitative evaluation of the biomarker, including, e.g., qualitative evaluation of the level or expression or change in level or expression of the biomarker. In some instances, a biomarker signature includes one or more quantitative measurements of a biomarker including e.g., measurements of the level of a biomarker or the expression level of a biomarker, including e.g., measurements of the absolute expression level of the biomarker, measurements of the relative expression level of the biomarker (e.g., relative to a second biomarker or a reference biomarker or reference biomarker level, etc.), measurements of the change in expression of a biomarker (e.g., the change in expression of a biomarker in response to a stimulus or the change in expression of a biomarker over time, etc.) and the like.

In some instances, a biomarker signature may include categorical measurements of one or more biomarkers. Categorical measurements of biomarkers of a biomarker signature may be qualitative or quantitative. In some instances, qualitative categorical measurements of biomarkers included in a biomarker signature are based on qualitative evaluations of biomarkers that are binned into categories based on binning criteria (e.g., present or absent; positive or negative; high or low; high, medium, or low; normal or abnormal; sufficient or deficient; detectable or undetectable; significant or not significant; not significant, significant, or very significant; etc.). In some instances, quantitative categorical measurements of biomarkers included in a biomarker signature are based on quantitative measurements of biomarkers that are binned into categories based on binning criteria (e.g., present or absent; positive or negative; high or low; high, medium, or low; normal or abnormal; sufficient or deficient; detectable or undetectable; significant or not significant; not significant, significant, or very significant; etc.). Binning criteria for categorizing biomarkers of a biomarker signature may vary and may be determined by any convenient means, including e.g., visual assessment of biomarker data, statistical assessment of biomarker data, empirical testing of biomarkers, hypothesis based testing of biomarkers or biomarker data, computer modeling of biomarker data, etc. In some instances, described in more detail elsewhere herein, binning is also referred to as thresholding and is used to categorize a biomarker as present or absent, high or low, or above or below a threshold.

In some embodiments, a biomarker signature includes a single evaluation or measurement of a biomarker for a particular sample, e.g., including a measurement of the amount of the biomarker present in the sample. For example, a biomarker signature may include a measurement of the amount of a particular protein biomarker present in a fluid sample, e.g., a blood sample of a subject. In some embodiments, a biomarker signature includes a plurality of evaluations or measurements of a biomarker for a particular sample, e.g., including a plurality of measurements of the level of a biomarker within a particular aspect of the sample. For example, a biomarker signature may include a plurality of measurements of the level of a biomarker present in or on the surface of a plurality of cells of a cellular sample, e.g., a blood sample.

In some instances, a biomarker signature may include a secondary measurement based on a plurality of primary measurements. Primary measurements may vary and include any and all individual biomarker measurements described herein. In some instances, primary measurements may include individual evaluations or measurements of biomarkers including, e.g., measurements of biomarkers within some aspect of a sample, including, e.g., measurements of the level of biomarkers of cells of a cellular sample. Secondary measurements may vary and will depend on the primary measurement or the plurality of primary measurements and in some instances include but are not limited to measurements of subgroups, subcategories, subpopulations and the like. In some instances, where a plurality of primary measurements represents the levels of a biomarker present in or on some aspect of a sample, a secondary measurement may include a quantification or categorization of the plurality of primary measurements. For example, where a plurality of primary measurements represents individual measurements of the levels of a particular biomarker for individual cells, a secondary measurement may represent further quantification of the biomarker levels of individual cells or, e.g., a categorization of the cells based on their individual biomarker level. Biomarker signatures, although not limited to primary and secondary measurements or combinations thereof, may include essentially only primary measurements, essentially only secondary measurements, or any combination of primary and secondary measurements.

In certain embodiments, a biomarker signature that includes measurements of more than one biomarker allows for an assessment or determination of higher confidence than the assessment or determination that could be made by analysis of the biomarkers independently. In some instances, a biomarker signature that includes measurements of more than one biomarker allows for an assessment or determination that could not be made by analysis of any of the individual biomarkers or any sub-combination of the biomarkers of the biomarker signature. Such biomarker signatures may in some instances be referred to or derived from multidimensional analysis. In some instances, multidimensional analysis is performed using a combination of biomarkers that have or have not been shown to be statistically significant in differentiating two or more different groups, e.g., treatment groups or patient groups. For example, in some instances a first biomarker may be used in combination with a second biomarker wherein the first biomarker has not been shown to statistically differentiate two different groups independently and the second biomarker has been shown to statistically differentiate two different treatment groups or patient groups independently, or vice versa. In some instances where two biomarkers are used in combination that do not independently statistically differentiate two different groups the combination of markers can statistically differentiate two different groups. In other instances where two biomarkers are used in combination that do independently differentiate, e.g., statistically differentiate, different groups the combination of markers can more significantly differentiate the different groups.

In certain instances, a biomarker signature may include one or more identified or evaluated or measured subgroups or subpopulations or proportions of a population of a particular sample having a shared characteristic or shared particular aspect that may vary within the sample. By “subgroup” or “subpopulation” or “proportion”, used interchangeably herein, is meant a portion of a larger group or a larger population of a sample that is differentiated from the larger group or larger population by one or more common characteristics or common aspects. For example, a subpopulation may share a common biomarker or characteristic or categorical biomarker level, including e.g., biomarker expression level. In some instances, a common characteristic or common aspect of a subgroup or subpopulation may be related to a shared biomarker, including but not limited to, e.g., shared presence or absence of a particular biomarker, shared level of a particular biomarker, shared expression of a particular biomarker, shared change in level of a particular biomarker, shared change in expression of a particular biomarker, etc. In some embodiments, a common characteristic or common aspect of a subgroup or subpopulation may be unrelated to a biomarker and may be some other aspect of the individual units of the subgroup or subpopulation. Other aspects, i.e. non-TB-biomarker aspects, of the individual units of the subgroup of subpopulation may vary and may be any convenient aspect of the individual units that may be determined, visualized, detected, measured, categorized, etc.

In certain instances, a population of which one or more subpopulations is a portion may be a population of cells, e.g., cells of a cellular sample or a portion of the cells of a cellular sample. Subpopulations of cells within a population may or may not be mutually exclusive, i.e., such subpopulations may or may not overlap and in some instances may overlap from 1% to 100%, including e.g., from 1% to 10%, from 10% to 20%, from 20% to 30%, from 30% to 40%, from 40% to 50%, from 50% to 60%, from 60% to 70%, from 70% to 80%, from 80% to 90%, from 90% to 100%, from 1% to 50%, from 50% to 100%, 90%, 95%, 100%, etc.

Cellular subpopulations of cells will vary in the common or shared aspects or characteristics which define particular subpopulations. In some instances, aspects which may define a cellular subpopulation include but are not limited to, e.g., cell size, cell shape, cell granularity, cell opacity, cell nuclear to cytoplasmic ratio, cellular contents (e.g., the presence or absence or amount of particular organelles or intercellular biomolecules or compounds (e.g., nucleic acid content, lipid content, carbohydrate content, etc.)), intercellular chemistry (e.g., intercellular pH), cellular surface contents (e.g., the presence or absence or amount of particular cell membrane components (e.g., cell surface proteins, cell surface lipids, cell surface carbohydrates, etc.)). In some instances, a cell subpopulation may be defined by the presence or absence or level of, including expression level of, or change in one or more particular biomarkers. In some embodiments, cells of a subpopulation having some level of expression of biomarker may be categorized based on a set threshold of biomarker expression as described elsewhere herein.

The difference in biomarker expression between two cells belonging to two different subpopulations of cells separated by a biomarker threshold, described below, or the mean difference in biomarker expression between two cell subpopulations will vary. In some instances, e.g., as measured in terms of the relative fluorescence of a particular biomarker as analyzed by flow cytometry, biomarker expression between two cells belonging to different subpopulations may range over 7 logs. For example, in some instances a cell of a first subpopulation may have a biomarker expression level, e.g., as detected using fluorescent reporters as described herein, that is different from the biomarker expression of a cell a second subpopulation by anywhere from 0.1 to 10⁷ times, including but not limited to, e.g., from 0.1 to 1 times, from 0.1 to 10 times, from 0.1 to 10² times, from 0.1 to 10³ times, from 0.1 to 10⁴ times, from 0.1 to 10⁵ times, from 0.1 to 10⁶ times, from 1 to 10 times, from 1 to 10² times, from 1 to 10³ times, from 1 to 10⁴ times, from 1 to 10⁵ times, from 1 to 10⁶ times, from 1 to 10⁷ times, from 10 to 10² times, from 10 to 10³ times, from 10 to 10⁴ times, from 10 to 10⁵ times, from 10 to 10⁶ times, from 10 to 10⁷ times, from 10² to 10³ times, from 10² to 10⁴ times, from 10² to 10⁵ times, from 10² to 10⁶ times, from 10² to 10⁷ times, from 10³ to 10⁴ times, from 10³ to 10⁵ times, from 10³ to 10⁶ times, from 10³ to 10⁷ times, from 10⁴ to 10⁵ times, from 10⁴ to 10⁶ times, from 10⁴ to 10⁷ times, from 10⁵ to 10⁶ times, from 10⁵ to 10⁷ times, and from 10⁶ to 10⁷ times.

In some instances, the number of cells having a biomarker level above or below a particular biomarker threshold level may be determined, e.g., to further determine the proportion of cells having a biomarker level above or below a particular threshold of a particular sample population. In certain instances, the size of a subpopulation of cells or the proportion cells of a particular sample population may be used in determining a biomarker signature and making a TB assessment. In some embodiments, the size of a single subpopulation of cells or a single proportion of cells of a particular sample population having a biomarker level above or below a particular threshold may constitute a biomarker signature. In other embodiments, the size of multiple subpopulations of cells or multiple proportions of cells of a particular sample population having biomarker levels above or below particular thresholds constitute a biomarker signature. The number of measured and/or identified subpopulations of cells of proportions of cells of a particular sample population used in producing a biomarker signature may vary and, in some instances, may range from 1 to 200, including, e.g., 1 to 100, 1 to 50, 1 to 20, 1 to 15, 1 to 10, 5 to 15, 2 to 10, 5 to 10, 7 to 10, 3 to 10, 3 to 7, 3 to 5, etc.

In some instances, in determining a biomarker signature one or more second subpopulations may be determined of one or more first subpopulations. For example, in some instances, a first subpopulation is determined that expresses a first biomarker above or below a particular threshold and a second subpopulation within the first subpopulation is determined that expresses a second biomarker above or below a particular threshold. Such analysis may be described in certain instances as biomarker co-expression and may be used to determine a subpopulation of cells expressing co-expressing two or more markers above or below certain threshold levels. In some instances a subpopulation may be determined that expresses a first biomarker above a certain threshold and a second marker below a certain threshold and may be described as, e.g., a cell subpopulation that is “positive” for a first marker and “negative” for a second marker. Also contemplated are subpopulations that are “double positive” or “double negative” accordingly. Such analysis is not limited to two subpopulation levels, i.e., two subpopulations, or two biomarkers and in some instances may consist of many subpopulation levels including a range of biomarkers. The number of subpopulations and biomarkers used in such analyses will vary and in some cases may be but is not limited to anywhere from 3 to 20, including e.g., 3 to 19, 3 to 18, 3 to 17, 3 to 16, 3 to 15, 3 to 14, 3 to 13, 3 to 12, 3 to 11, 3 to 10, 3 to 9, 3 to 8, 3 to 7, 3 to 6, 3 to 5, and 3 to 4. Accordingly, subpopulations may have any combination of presence or absence of biomarkers above or below particular threshold levels, including e.g., “positive” for a first biomarker, “negative” for a second biomarker and “positive” for a third biomarker, or “triple positive” or “triple negative”, etc. Such analysis is not limited to distinct subpopulations and in some instances subpopulations may overlap or a subpopulation may not be entirely contained within one or more higher level subpopulations.

Cellular samples from which a subpopulation of cells may be identified or evaluated or measured may vary and include any sample obtained from a subject that contains cells. Cellular samples may be obtained in any convenient manner including but not limited to, e.g., tissue biopsy, including punch biopsy, bone marrow biopsy and by taking blood, bronchial aspirate, cerebrospinal fluid, sputum or other body fluids. In some instances, a cellular sample used in identifying a subpopulation or producing a biomarker signature or making a TB assessment may be unprocessed or taken directly from the subject and used in analysis, including e.g., a whole blood sample. In other instances, a cellular sample may be obtained by processing a sample obtained from a patient, including e.g., isolating cells from the sample, concentrating the cells of the sample, dissociating the cells of the sample. In some instances, the cellular sample is a blood sample or a processed blood sample, including e.g., a preparation of peripheral blood mononuclear cells (PBMCs), a preparation of serum, a preparation of immune cells, etc.

In some embodiments, the cellular sample may be obtained from a naïve subject or a subject that has not had any prior medical or pharmacological intervention, e.g., not had any medical or pharmacological intervention related to a disease assessment or diagnosis or treatment, including e.g., a TB assessment or TB treatment. In some embodiments, the subject may be a treated patient or a patient that has had some amount of prior medical or pharmacological intervention, including e.g., treatment for a disorder, e.g., a lung disorder, or an infection, e.g., a TB infection, or TB treatment, including e.g., those TB treatments described herein.

In some instances, a biomarker signature or an assessment, including monitoring and diagnosing, of TB, as described herein, may be performed using antigen-stimulated samples. Antigen stimulation may be performed before sample collection, e.g., antigen stimulation may be performed in the subject by contacting the subject with the antigen a subsequently collecting the sample after antigen stimulation, or after sample collection, e.g., antigen stimulation may be performed in culture after the cells of the sample have been isolated from the subject. Antigen stimulation is performed using any useful antigen including Mycobacterium tuberculosis (MTB) antigens known to elicit an antigenic response. In some instances, a potential MTB antigen, i.e. an antigen derived from Mycobacterium tuberculosis but not necessarily known to be antigenic, may be tested for an antigenic response by use in antigen simulation of a sample and analysis for the presence of stimulation. Methods useful in detecting antigen stimulation include those presented herein for detecting differences in biomarker signatures as well as those conventionally used in the art. In other instances, conventional antigen stimulation analysis is used in parallel with method of host TB biomarker analysis as described herein. For example, in one embodiment, antigen stimulated samples are processed and surface host TB biomarker expression is analyzed as described herein in parallel with analysis of sample supernatant for the levels non-cell-associated biomarkers, e.g., soluble host biomarkers, including e.g., cytokines and chemokines. In some instances, parallel analysis of cellular host TB biomarkers and non-cell-associated biomarkers allows for the correlations of two or more TB analyses in order to, e.g., increase the accuracy or confidence of a subsequent TB assessment.

In some instances, a TB assessment, as described herein, making use of an antigen stimulated sample may include an assessment or a correction of one or more TB host biomarkers showing differential expression in antigen stimulated samples as compared to unstimulated samples. For example, in some instances, a TB assessment of an antigen stimulated sample may include an assessment of one or more TB host biomarkers, including, e.g., identification of a cellular subpopulation having expression of a TB host biomarker above or below a threshold level, that has been shown to be differentially expressed in antigen stimulated samples including but not limited to, e.g., CD41a, CD45Ra, CD61, CD4v4, CD49a, CD62L, and the like

In instances, where a TB assessment configured for unstimulated samples includes detection of a TB host biomarker that is differentially expressed in antigen stimulated samples the detection of the differentially expressed TB host biomarker may be adjusted (e.g., corrected) based on the differential expression in the stimulated sample as compared to that of an unstimulated sample. In some instances, where adjustment of TB host biomarker detection is performed, such adjustments may be based on reference measurements, including but not limited to, e.g., an antigen stimulated reference, an unstimulated reference, or combinations thereof. In instances, where the differential expression of a TB host biomarker is shown to not be significant, e.g., statistically significant, such a lack of significance may indicate that one or more adjustments for antigen stimulated samples is unnecessary and antigen stimulated samples may be assessed according to methods used for unstimulated samples.

In some instances, samples used in making a TB assessment are fresh samples, e.g., samples collected from subject within 1 to 5 days, including e.g., within 5 days, within 4 days, within 3 days, within 2 days, and within 1 day. In some instances, samples used in making a TB assessment are previously collected samples. Previously collected samples may be stored under appropriate conditions before analysis and may be processed, e.g., partitioned, including e.g., the removal or partitioning of a particular component or portion of a blood sample, or unprocessed prior to storage. In some instances, appropriate storage conditions include refrigerator storage, including e.g., storage below room temperature but above freezing temperatures, including e.g., storage between 21° C. and 1° C., between 10° C. and 1° C., between 10 and 4° C., etc. Refrigeration may, in some instances, include sample storage on ice. In some instances, appropriate storage conditions include freezing conditions, including e.g., freezing at temperatures ranging from 0° C. to −200° C., including, e.g., storage at 0° C. to −10° C., 0° C. to −20° C., −20° C. to −50° C., −20° C. to −60° C., −20° C. to −70° C., −60° C. to −80° C., −60° C. to −90° C., −60° C. to −100° C., −60° C. to −110° C., −60° C. to −120° C., −120° C. to −130° C., −120° C. to −140° C., −120° C. to −150° C., −120° C. to −160° C., −120° C. to −170° C., −120° C. to −180° C., −120° C. to −190° C., −120° C. to −200° C., etc.

In certain instances, biomarker detection involves the evaluation or assessment of the level of a biomarker. The level of a biomarker may, in some instances, refer to the expression level of a biomarker. By “expression level of a biomarker” or “biomarker expression” is meant the level at which a particular biomarker is present in a sample and may include but is not limited to, e.g., the level of a soluble biomarker in a bodily fluid, the level of a cellular biomarker present in a sample, the level of a cellular biomarker present within a cell, the level of a cellular biomarker present on a cell, the level of a cellular biomarker present on the surface of a cell, the level of a cellular biomarker present on a cellular membrane. In some instances, expression level of a biomarker may refer to the relative abundance of RNA, DNA or protein abundances or activity levels. Expression level of a biomarker may be evaluated or determined or measured by any convenient method including but not limited to, e.g., gene-chips, gene arrays, beads, multiplex PCR, quantitiative PCR, run-on assays, Northern blot analysis, Western blot analysis, protein expression, fluorescence activated cell sorting (FACS), enzyme linked immunosorbent assays (ELISA), chemiluminescence studies, enzymatic assays, or any other method, apparatus and system for the determination and/or analysis of expression that are readily commercially available.

In certain embodiments biomarker detection and/or measurement of biomarker levels is performed using flow cytometry. Flow cytometry is a technique for counting, examining, and sorting microscopic particles suspended in a stream of fluid. It allows simultaneous multi-parametric analysis of the physical and/or chemical characteristics of single cells flowing through an optical and/or electronic detection apparatus. Fluorescence-activated cell sorting (FACS) is a specialized type of flow cytometry. FACS provides a method for sorting a heterogeneous mixture of biological cells into two or more containers, generally one cell at a time, based upon the specific light scattering and fluorescent characteristics of each cell. The flow cytometer and the FACS machine are useful scientific instruments as they provide fast, objective and quantitative recording of signals, e.g., fluorescent signals, and/or detection of cellular characteristics, e.g., size, granularity, viability, etc., from individual cells as well as physical separation of cells of particular interest. Fluorescent signals used in flow cytometry, for instance when quantifying and/or sorting cells by any marker present on or in the cell, typically are fluorescently-tagged antibody preparations or fluorescently-tagged ligands for binding to antibodies or other antigen-, epitope- or ligand-specific agent, such as with biotin/avidin binding systems or fluorescently-labeled and optionally addressable beads (e.g. microspheres or microbeads). The markers or combinations of markers detected by the optics and/or electronics of a flow cytometer vary and in some cases include but are not limited to: cell surface markers, intracellular and nuclear antigens, DNA, RNA, cell pigments, cell metabolites, protein modifications, transgenic proteins, enzymatic activity, apoptosis indicators, cell viability, cell oxidative state, etc.

In certain instances, flow cytometry is performed using a detection reagent, e.g., a fluorochrome-labeled antibody, e.g., a monoclonal antibody, with specific avidity against a biomarker antigen of interest of a cell, e.g., a biomarker present on the surface of a cell. A cellular sample is contacted with a detection reagent under conditions sufficient to allow the detection reagent to bind the biomarker antigen and the cells of the sample are loaded into the flow cytometer, e.g., by loading the entire sample or a portion of the unmodified sample into the flow cytometer or by first isolating the cells from the cellular sample using cell isolation methods known in the art or described herein and re-suspending the isolated cells in a suitable buffer, e.g., running buffer. The cells loaded into the flow cytometer are run through the flow cytometer, e.g., by flowing cell containing buffer or liquid sample through the flow cell of the flow cytometer. The flow cytometer detects events as the cell passes one or more detection areas of the flow cytometer. For example, the flow cytometer may detect fluorescence emitted from a fluorochrome of a detection reagent upon excitation of the fluorochrome with a particular wavelength of light. In some instances, the flow cytometer detects the relative intensity of a particular signal, e.g., fluorescence of a particular detection reagent, of a particular cell, e.g., the quantify the level of a marker present on the surface of the cell and/or to qualitatively categorize the cell, e.g., as a cell that is positive for a particular marker or a cell that is negative for a particular marker. Detected events are counted or otherwise evaluated by the flow cytometer without or without input from an operator and used to determine, e.g., the total number of cells, the number or proportion of cells bound to a particular detection reagent, the overall presence or amount of a particular feature of a cell population, etc. detection reagents useful in flow cytometry, e.g., including but not limited to antibodies, may be created in the laboratory using well established methods and are commercially available for e.g., BD (Franklin Lakes, N.J.) and BD Biosiences (San Jose, Calif.).

In some instances, a biomarker threshold is determined by making a comparison of the levels of biomarker expression in two separate populations of cells known to differ in their expression of the subject biomarker. For example, a first cell population known to express a high level of Biomarker X is measured, e.g., on a flow cytometer, and compared to a second cell population, known to express a low level of Biomarker X and the comparison is used to determine a threshold level that may be used to categorize cells as either having a low or a high level of expression of Biomarker X.

In some instances, a biomarker threshold is determined by making a comparison of the levels of biomarker expression within a population of cells, e.g., a population of cells of unknown expression levels of Biomarker X or a population of cells suspected of containing subpopulations of cells having different expression levels of Biomarker X. For example, the expression level of Biomarker X is measured on a flow cytometer of at least a sufficient number of cells such that the measurements may be plotted, e.g., on a histogram, and separation between two or more subpopulations of cells is revealed based on individual cell expression levels of Biomarker X. Accordingly, the flow cytometer operator may then determine a threshold level between the subpopulations that may be used to categorize cells as belonging to a particular subpopulation, e.g., a subpopulation having a low level of expression of Biomarker X or a subpopulation having high level of expression of Biomarker X.

In some instances, the biomarker threshold is based on the limit of detection of the flow cytometer. For example, cells of a population of cells may be identified as expressing a particular biomarker (i.e. being positive for a particular biomarker) if the cells have any detectable level of a particular biomarker. Likewise, cells of a population of cells may be identified as not expressing a particular biomarker (i.e. being negative for a particular biomarker) if the cells do not have a detectable level of a particular biomarker. Accordingly, the detection level of the flow cytometer may be used to determine the biomarker threshold.

In some instances, the biomarker threshold is based on previously determined biomarker expression levels (i.e. reference biomarker levels), e.g., from previously performed control experiments or previously acquired reference expression levels. For example, biomarker expression levels determined in previously analyzed patient samples, e.g., from TB patients and healthy patients such as those described herein, may be used to determine biomarker threshold levels. In some instances, biomarker expression levels expected of cells obtained from healthy subjects may be used to determine normal biomarker expression levels such that a biomarker threshold that is representative of the normal biomarker expression range may be determined. In such instances, biomarker expression outside, i.e., above or below, the normal biomarker expression range is considered to be either above or below the particular biomarker threshold. In some instances, use of such previously determined biomarker expression levels or previously determined threshold levels allows analysis of cells and the identification of cellular subpopulations in the absence of a control or reference cellular sample.

In some aspects of the present disclosure, biomarkers are provided for making a TB assessment and for use in producing a biomarker signature for making a TB assessment. By “biomarker”, or in some instances simply “marker”, is meant any molecular, chemical, or physiological factor whose representation in a sample is associated with a clinical phenotype or clinical outcome. For example, a TB biomarker may be differentially represented in a sample of a subject having TB as compared to a healthy individual or a subject having TB as compared to a subject having a non-TB lung disease or a subject responding to TB therapy as compared to a subject not responding to TB therapy or a subject requiring TB therapy as compared to a subject not requiring TB therapy or a subject requiring further TB therapy as compared to a subject not requiring further TB therapy, etc.

Specific agents that may be evaluated as biomarkers include but are not limited to, e.g., polypeptides (e.g., peptides, proteins, lipoproteins, etc.), carbohydrates, lipids, metabolites, amino acids, electrolytes, nucleic acids (e.g., DNA, mRNA, microRNA, etc.) and the like. Biomarkers useful in assessing TB or supplementing TB assessments may be associated with cells, i.e., “cellular biomarkers” or not associated with cells, i.e., “non-cell-associated biomarkers”. In some instances, non-cell-associated biomarkers include soluble host biomarkers, e.g., host serum markers. By “host serum markers” is meant those markers present in a subject's serum that may be used to diagnose disease or infection, assess disease state, or monitor disease progression or treatment efficacy. In certain instances, additional biomarkers may include subject or patient characteristics, including e.g., physiological characteristics (e.g., blood volume, blood pressure, heart rate, blood pH, blood oxygen, oxygen consumption, respiratory rate, basal metabolism, body temperature, water balance, urine density, proteinuria, aminoaciduria, creatinuria, etc.) or behavioral characteristics (e.g., verbal function, vision function, olfactory function, auditory function, tactile function, memory function, mobility, etc.). The presence, absence, or level (e.g., high level or low level) of a particular additional biomarker or a change in a particular biomarker, including e.g., a change in biomarker level or biomarker expression (i.e. increased level or expression or decreased level or expression), as included in TB assessments, may be correlated with a particular TB diagnosis or clinical evaluation. Such additional biomarkers are described in greater detail below.

Those biomarkers expressed by a host, e.g., expressed by host cells or expressed on host cells, may be referred to as host biomarkers. In some instances, host biomarkers that are differentially expressed by a host infected with TB or a subject having TB disease as compared to a non-infected subject or a subject not having TB disease are referred to as TB host biomarkers. TB host biomarkers may be detected, measured, or evaluated by any convenient method, including those methods described for biomarkers previously.

Subject biomarkers useful in making an assessment of the present disclosure include, e.g., cytokines, cytokine receptors, and markers of inflammation. Cytokines and cytokine receptors are important for cell signaling to influence the behaviors of other cells but are generally not hormones or growth factors. In some instances cytokines or their receptors that are useful as biomarkers include but are not limited to chemokines, interferons, interleukins, lymphokines, tumor necrosis factor, and the like. Such cytokines are produced in a wide range of different cells including, but not limited to, immune cells, macrophages, B lymphocytes, T lymphocytes, mast cells, and the like. Such cytokines are also produced in non-immune cells or cells that are not necessarily immune cells, e.g., endothelial cells, fibroblasts, stromal cells and the like.

In certain embodiments, TB host biomarkers include markers or combinations of markers detected by the optics and/or electronics of a flow cytometer. In some instances such markers are surface antigens, e.g., proteins, expressed or displayed on the surface of a cell and used to identify a subpopulation of cells based on similar expression levels of the same marker or markers. In other instances such markers are cellular characteristics that can be detected by the optics and/or electronics of a flow cytometer, as described herein. Markers of interest include but are not limited to those listed in Tables 1-3. Markers of interest include those markers described herein that show significantly different expression levels in various treatment groups, e.g., groups at various time points following the initiation of treatment, and control groups, e.g., healthy controls or controls having other lung diseases, after Bonferroni correction, those that show significantly different expression levels in various treatment and control groups by any statistical method used herein, and those that show expression trends across treatment and/or control groups regardless of statistical significance.

In some instances, biomarkers useful in determining a biomarker signature for diagnosing TB in a subject are those TB host biomarkers that are expressed above or below a threshold level in a subpopulation of cells of a cellular sample obtained from subjects suspected of having TB. For example, in some instances the expression level of a TB host biomarker is measured and used to determine the relative size of a subpopulation of cells of a cellular sample obtained from a subject suspected of having TB and the size of the subpopulation is compared to a healthy control reference. In some embodiments, the relative size of the subpopulation of cells having expression of the TB host biomarker above a particular threshold in subjects suspected of having TB is smaller than that of the reference standard.

In some instances, biomarkers useful in determining a biomarker signature for diagnosing TB in a subject are those TB host biomarkers that are expressed above or below a threshold level in a subpopulation of cells of a cellular sample obtained from subjects suspected of having TB. For example, in some instances the expression level of a TB host biomarker is measured and used to determine the relative size of a subpopulation of cells of a cellular sample obtained from a subject suspected of having TB and the size of the subpopulation is compared to a healthy control reference. In some embodiments, the relative size of the subpopulation of cells having expression of the TB host biomarker above a particular threshold in subjects suspected of having TB is larger than that of the reference standard, e.g., including but not limited to CD126 and fMLP r.

In some instances, biomarkers useful in determining a biomarker signature for diagnosing TB in a subject having other lung diseases are those TB host biomarkers that are expressed above or below a threshold level in a subpopulation of cells of a cellular sample obtained from subjects suspected of having TB. For example, in some instances the expression level of a TB host biomarker is measured and used to determine the relative size of a subpopulation of cells of a cellular sample obtained from a subject suspected of having TB and the size of the subpopulation is compared to a reference standard derived from non-TB subjects having other lung diseases. In some embodiments, the relative size of the subpopulation of cells having expression of the TB host biomarker above a particular threshold in subjects suspected of having TB is smaller than that of the reference standard.

In some instances, biomarkers useful in determining a biomarker signature for diagnosing TB in a subject having other lung diseases are those TB host biomarkers that are expressed above or below a threshold level in a subpopulation of cells of a cellular sample obtained from subjects suspected of having TB. For example, in some instances the expression level of a TB host biomarker is measured and used to determine the relative size of a subpopulation of cells of a cellular sample obtained from a subject suspected of having TB and the size of the subpopulation is compared to a reference standard derived from non-TB subjects having other lung diseases. In some embodiments, the relative size of the subpopulation of cells having expression of the TB host biomarker above a particular threshold in subjects suspected of having TB is larger than that of the reference standard, e.g., including but not limited to CD120b and CD126.

In some instances, biomarkers useful in determining a biomarker signature for assessing or diagnosing TB in a patient in an early phase of TB treatment, e.g., after four weeks of treatment, are those TB host biomarkers that are expressed above or below a threshold level in a subpopulation of cells of a cellular sample obtained from TB treatment patients. For example, in some instances the expression level of a TB host biomarker is measured and used to determine the relative size of a subpopulation of cells of a cellular sample obtained from a patient in an early phase of TB treatment and the size of the subpopulation is compared to a reference standard derived from a healthy control reference.

In some instances, biomarkers useful in determining a biomarker signature for assessing or diagnosing TB in a patient in an early phase of TB treatment, e.g., after four weeks of treatment, and having other lung disease are those TB host biomarkers that are expressed above or below a threshold level in a subpopulation of cells of a cellular sample obtained from TB treatment patients. For example, in some instances the expression level of a TB host biomarker is measured and used to determine the relative size of a subpopulation of cells of a cellular sample obtained from a patient in an early phase of TB treatment and having other lung disease and the size of the subpopulation is compared to a reference standard derived from non-TB subjects having other lung diseases.

In some instances, biomarkers useful in determining a biomarker signature for assessing or diagnosing TB in a patient in a late phase of TB treatment, e.g., after 24 weeks of treatment, are those TB host biomarkers that are expressed above or below a threshold level in a subpopulation of cells of a cellular sample obtained from TB treatment patients. For example, in some instances the expression level of a TB host biomarker is measured and used to determine the relative size of a subpopulation of cells of a cellular sample obtained from a patient in a late phase of TB treatment and the size of the subpopulation is compared to a reference standard derived from a healthy control reference.

In some instances, biomarkers useful in determining a biomarker signature for assessing or diagnosing TB in a patient in a late phase of TB treatment, for example after 24 weeks of treatment, and having other lung disease are those TB host biomarkers that are expressed above or below a threshold level in a subpopulation of cells of a cellular sample obtained from TB treatment patients. For example, in some instances the expression level of a TB host biomarker is measured and used to determine the relative size of a subpopulation of cells of a cellular sample obtained from a patient in a late phase of TB treatment and having other lung disease and the size of the subpopulation is compared to a reference standard derived from non-TB subjects having other lung diseases.

In some instances, biomarkers useful in determining a biomarker signature for monitoring TB treatment in a patient in an early phase of TB treatment, e.g., after 4 weeks of treatment, are those TB host biomarkers that are expressed above or below a threshold level in a subpopulation of cells of a cellular sample obtained from TB treatment patients. For example, in some instances the expression level of a TB host biomarker is measured and used to determine the relative size of a subpopulation of cells of a cellular sample obtained from a patient in an early phase of TB treatment and the size of the subpopulation is compared to a reference standard derived from untreated TB subjects. In some embodiments, the relative size of the subpopulation of cells having expression of the TB host biomarker above a particular threshold in patients in an early phase of TB treatment is smaller than that of the reference standard, e.g., including but not limited to CCR7, CD120b, CD126, CD28, CD4, CD4 v4 and CD62L.

In some instances, biomarkers useful in determining a biomarker signature for monitoring TB treatment in a patient in an early phase of TB treatment, e.g., after 4 weeks of treatment, are those TB host biomarkers that are expressed above or below a threshold level in a subpopulation of cells of a cellular sample obtained from TB treatment patients. For example, in some instances the expression level of a TB host biomarker is measured and used to determine the relative size of a subpopulation of cells of a cellular sample obtained from a patient in an early phase of TB treatment and the size of the subpopulation is compared to a reference standard derived from untreated TB subjects. In some embodiments, the relative size of the subpopulation of cells having expression of the TB host biomarker above a particular threshold in patients in an early phase of TB treatment is larger than that of the reference standard, e.g., including but not limited to CD58.

In some instances, biomarkers useful in determining a biomarker signature for monitoring TB treatment in a patient in a late phase of TB treatment, e.g., after 24 weeks of treatment, are those TB host biomarkers that are expressed above or below a threshold level in a subpopulation of cells of a cellular sample obtained from TB treatment patients. For example, in some instances the expression level of a TB host biomarker is measured and used to determine the relative size of a subpopulation of cells of a cellular sample obtained from a patient in a late phase of TB treatment and the size of the subpopulation is compared to a reference standard derived from untreated TB subjects. In some embodiments, the relative size of the subpopulation of cells having expression of the TB host biomarker above a particular threshold in patients in a late phase of TB treatment is smaller than that of the reference standard, e.g., including but not limited to CCR7, CD120b, CD126, CD28, CD4, CD4 v4, and CD62L.

In some instances, biomarkers useful in determining a biomarker signature for monitoring TB treatment in a patient in a late phase of TB treatment, e.g., after 24 weeks of treatment, are those TB host biomarkers that are expressed above or below a threshold level in a subpopulation of cells of a cellular sample obtained from TB treatment patients. For example, in some instances the expression level of a TB host biomarker is measured and used to determine the relative size of a subpopulation of cells of a cellular sample obtained from a patient in a late phase of TB treatment and the size of the subpopulation is compared to a reference standard derived from untreated TB subjects. In some embodiments, the relative size of the subpopulation of cells having expression of the TB host biomarker above a particular threshold in patients in a late phase of TB treatment is larger than that of the reference standard, e.g., including but not limited to CCR6, CD107a, CD44, CD45RB, and CD58.

In some instances, biomarkers useful in determining a biomarker signature for monitoring TB treatment progression in a patient during TB treatment are those TB host biomarkers that are expressed above or below a threshold level in a subpopulation of cells of a cellular sample obtained from TB treatment patients at various time points during treatment. For example, in some instances the expression level of a TB host biomarker is measured and used to determine the relative size of a subpopulation of cells of a cellular sample obtained from a patient in a late phase of TB treatment and the size of the subpopulation is compared to a reference standard derived from TB subjects in an early phase of TB treatment or vice versa. In some embodiments, the relative size of the subpopulation of cells having expression of the TB host biomarker above a particular threshold in patients in a late phase of TB treatment is smaller than that of the reference standard for patients in an early phase of treatment, e.g., including but not limited to CCR7, CD120b, CD126, CD28, CD4, CD4 v4, and CD62L.

In some instances, biomarkers useful in determining a biomarker signature for monitoring TB treatment progression in a patient during TB treatment are those TB host biomarkers that are expressed above or below a threshold level in a subpopulation of cells of a cellular sample obtained from TB treatment patients at various time points during treatment. For example, in some instances the expression level of a TB host biomarker is measured and used to determine the relative size of a subpopulation of cells of a cellular sample obtained from a patient in a late phase of TB treatment and the size of the subpopulation is compared to a reference standard derived from TB subjects in an early phase of TB treatment or vice versa. In some embodiments, the relative size of the subpopulation of cells having expression of the TB host biomarker above a particular threshold in patients in a late phase of TB treatment is larger than that of the reference standard for patients in an early phase of treatment, e.g., including but not limited to CD58.

In some instances, biomarkers useful in determining a biomarker signature for assessing the likelihood of a particular patient outcome or determining a course of TB treatment in a patient, e.g., at the time of diagnosis, are those TB host biomarkers that are differentially expressed between TB patients having different post treatment outcomes, e.g., positive or negative outcomes. Such TB host biomarkers may be differentially expressed in a subpopulation of cells of a cellular sample obtained from the TB patients at an early stage of disease or treatment (e.g., at baseline). For example, in some instances the expression level of a TB host biomarker is measured and used to determine the relative size of a subpopulation of cells of a cellular sample obtained from a patient at an early stage of disease or treatment and the size of the subpopulation is compared to a reference standard derived from TB subjects for which treatment outcomes are known. Any convenient method may be used for determining TB patient treatment outcome including but not limited to those clinical evaluations and/or tests described herein, including e.g., clinical diagnosis, PET scan, combined PET-CT scan, and the like. In some embodiments, the relative size of the subpopulation of cells having expression of the TB host biomarker above a particular threshold in patients in an early stage of disease or TB treatment is significantly different than that of a reference standard derived from TB patients with negative treatment outcomes (e.g., clinically diagnosed as not cured, PET scan not improved, combined PET-CT scan poor, etc.) e.g., including but not limited to CD18, CD11a, CD50, CD48, CD53, CD62P, CD81, CD45RO, and CD4v4. In some embodiments, the relative size of the subpopulation of cells having expression of the TB host biomarker above a particular threshold in patients in an early stage of disease or TB treatment is significantly different than that of a reference standard derived from TB patients with positive treatment outcomes (e.g., clinically diagnosed as cured, PET scan improved, combined PET-CT scan good, etc.) e.g., including but not limited to CD18, CD11a, CD50, CD48, CD53, CD62P, CD81, CD45RO, and CD4v4.

In certain embodiments, an assessment is made based on a biomarker signature that includes biomarkers in addition to TB host biomarkers as described herein. In certain instances, such additional biomarkers may be referred to as “non-TB host biomarkers” or simply “additional biomarkers”. Any convenient non-TB host biomarkers or additional biomarkers useful in making an assessment of a subject suspected of having TB or known to have TB, including for e.g., biomarkers used to assess general health or a non-TB related condition or disease may find use in the assessments described herein.

In certain embodiments where an assessment is made based on a biomarker signature that includes biomarkers in addition to TB host biomarkers as described herein, such additional biomarkers may include gene expression changes, e.g., gene expression changes within host cells, e.g., host blood cells, identified by assaying the relative amount of mRNA for particular genes in cells obtained from different treatment groups or patient groups. For example, genes that are differentially expressed in pulmonary TBs patients assayed at various points of treatment, e.g., at diagnosis and during treatment, including but not limited to those described by Cliff et al. (2013) J. Infect. Dis. 207(1):18-29, the disclosure of which is incorporated herein by reference.

In some instances, certain biomarkers have characteristics that justify their exclusion from a particular biomarker signature or TB assessment. Characteristics of biomarkers that may justify exclusion from a biomarker signature include but are not limited to, e.g., high baseline expression of the biomarker, low baseline expression of the biomarker, variable expression of the biomarker in control samples, etc. For example, in some instances, biomarkers useful in determining a biomarker signature for monitoring TB treatment progression in a patient during TB treatment specifically excludes host TB biomarkers that are expressed at high levels. For example, in some instances biomarkers that show a statistically significant difference between two treatment groups may be excluded from a biomarker signature used to make a TB assessment, e.g., because such difference is not biologically meaningful. Host TB biomarkers that are expressed at high levels may vary and in some instances include but are not limited to those markers that are present in 85% to 100% of the measured population of cells, including e.g., 86% to 100%, 87% to 100%, 88% to 100%, 89% to 100%, 90% to 100%, 91% to 100%, 92% to 100%, 93% to 100%, 94% to 100%, 95% to 100%, 96% to 100%, 97% to 100%, 98% to 100%, 99% to 100%, 85% to 99%, 90% to 99%, and 95% to 99%.

The subject disclosure describes biomarker evaluations and/or measurements that are used to produce a biomarker signature which may be used in making a TB assessment. The use of biomarker evaluations and/or measurements and produced biomarker signatures in making TB assessments will vary as described herein. In certain embodiments, TB assessments of subjects, e.g., for use in diagnosing TB or clinically monitoring TB in a subject, are performed by detecting the levels of host TB biomarkers, e.g., TB host biomarkers including TB host biomarkers present on the surface of cells, obtained from the subject. For example, the level of a host biomarker used in making an assessment of a subject may be measured and compared to a particular biomarker threshold, e.g., to determine whether the biomarker is present above a particular threshold level or below a particular threshold level. In certain instances, the number or proportion of cells of a sample having a biomarker level above or below a particular biomarker threshold level is determined, e.g., to identify a particular subpopulation or multiple subpopulations or produce a biomarker signature, and used in making the assessment.

The use of biomarker signatures in making TB assessments will vary and may depend on the particular subject or patient population and the purpose of the particular TB assessment. Biomarker signatures may be compared to reference biomarker signatures to guide diagnosis or treatment or monitoring of treatment or monitoring of disease progression and the particular aspects of the TB assessment may depend on the medical history or treatment circumstances of the particular subject.

In certain embodiments, persons with latent TB infection may remain untreated and the TB infection may be monitored using the assessments described herein, e.g., an untreated TB infected subject may be monitored in order to detect or predict the development of TB disease. In other instances, persons with latent TB infection may be treated, e.g., to prevent the development of TB disease, and the TB infection may be monitored using the assessments described herein, e.g., a TB infected subject undergoing treatment may be monitored in order to detect or predict the development of TB disease. When used for monitoring, e.g., monitoring TB infection or TB disease, the frequency of TB assessments may vary and may range, e.g., from frequencies of daily to annually, including but not limited to daily, every other day, every two days, twice weekly, weekly, once every other week, once every three weeks, monthly, once every two months, quarterly, once every four months, once every five months, once every six months, once every seven months, once every eight months, once every nine months, once every ten months, once every eleven months, annually, etc. In some instances, the frequency of monitoring may be based on a subjects risk of developing TB disease, e.g., subjects with higher risk of developing TB disease, e.g., immunocompromised subjects, may undergo monitoring with high assessment frequency and subjects with normal immune function, e.g., non-immunocompromised subjects, may undergo monitoring with assessment low frequency.

In certain instances, TB assessments as described herein may be used to monitor TB treatment, e.g., TB treatment of subjects with latent TB infection or subjects with TB disease. Treatments of TB vary, as described below, during which time monitoring may be performed at some regular or variable frequency, and in some cases may include taking one or more TB affective drugs for a period of time, e.g., ranging from one month to many years, including but not limited to, e.g., 1 to 12 months, 2 to 12 months, 3 to 12 months, 4 to 12 months, 5 to 12 months, 6 to 12 months, 1 to 9 months, 2 to 9 months, 3 to 9 months, 4 to 9 months, 5 to 9 months, 6 to 9 months, 9 months to 12 months, 1 year to 2 years, 1 year to 3 years, etc. In some instances, one or more TB assessments are performed at or near the planned end of treatment including but not limited to on the last planned day of treatment or within 1 day to 1 month of the planned last day of treatment, including e.g., within 1 to 2 days, within 2 to 3 days, within 3 to 5 days, within a week, within 2 weeks, within 3 weeks, within a month, etc., in order to determine whether treatment should be stopped as planned. For example, in some instances a TB assessment performed at or near the planned end of treatment (i.e. an end-of-treatment assessment) may indicate that treatment should not be stopped as planned, e.g., the TB assessment may indicate, e.g., a higher than anticipated state of TB infection or TB disease such that a medical professional would determine that treatment should be continued. In other instances, e.g., an end-of-treatment assessment may indicate that treatment should be stopped sooner than planned, e.g., the TB assessment may indicate, e.g., a lower than anticipated state of TB infection or TB disease such that a medical professional would determine that treatment should be stopped.

Current treatments of TB vary and particular TB treatment regimens are chosen by medical practitioners depending upon a number of clinical factors including but not limited to, e.g., characteristics of the particular subject undergoing therapy, characteristics of the particular TB being treated, e.g., TB disease, latent TB, drug-resistant TB, etc. For example, those current treatments suggested by the Center for Disease Control (CDC) for latent TB and TB disease include those described in Table 5 and Table 6 below:

TABLE 5
Latent TB Infection Treatment Regimens

| Drugs | Duration | Interval | Minimum doses |
|---|---|---|---|
| Isoniazid | 9 months | Daily | 270 |
| | | Twice weekly* | 76 |
| Isoniazid | 6 months | Daily | 180 |
| | | Twice weekly* | 52 |
| Isoniazid and Rifapentine | 3 months | Once weekly* | 12 |
| Rifampin | 4 months | Daily | 120 |

*Use Directly Observed Therapy (DOT)

TABLE 6
Basic TB Disease Treatment Regimens

| Preferred Regimen | Alternative Regimen | Alternative Regimen |
|---|---|---|
| Initial Phase | Initial Phase | Initial Phase |
| Daily INH, RIF, PZA, and | Daily INH, RIF, PZA, and EMB* for 14 | Thrice-weekly INH, RIF, |
| EMB* for 56 doses (8 | doses (2 weeks), then twice weekly | PZA, and EMB* for 24 |
| weeks) | for 12 doses (6 weeks) | doses (8 weeks) |
| Continuation Phase | Continuation Phase | Continuation Phase |
| Daily INH and RIF for | Twice-weekly INH and RIF for 36 | Thrice-weekly INH and RIF |
| 126 doses (18 weeks) | doses (18 weeks) | for 54 doses (18 weeks) |
| or | | |
| Twice-weekly INH and | | |
| RIF for 36 doses (18 | | |
| weeks) | | |

Abbreviations:
isoniazid (INH),
rifampin (RIF),
pyrazinamide (PZA),
ethambutol (EMB).
*EMB can be discontinued if drug susceptibility studies demonstrate susceptibility to first-line drugs.
Note:
A continuation phase of once-weekly INH/rifapentine can be used for HIV negative patients who do not have cavities on the chest film and who have negative acid-fast bacilli (AFB) smears at the completion of the initial phase of treatment.

As indicated above, in some instances TB treatment may include a continuation phase. In certain instances the continuation phase of treatment is given for a period of time following an initial phase of treatment, e.g., for 4 or 7 months. The length of the continuation phase may vary and may depend upon particular patient characteristics. For example, a 7-month continuation phase is recommended for particular patient groups, including e.g., patients with cavitary pulmonary tuberculosis caused by drug-susceptible organisms and whose sputum culture obtained at the time of completion of 2 months of treatment is positive; patients whose initial phase of treatment did not include PZA; and patients being treated with once weekly INH and rifapentine and whose sputum culture obtained at the time of completion of the initial phase is positive. In certain embodiments, monitoring of treatment through the use of TB assessments described herein may be performed during such a continuation phase. In other instances monitoring of treatment through the use of TB assessments described herein may be stopped before or during a continuation phase.

The end of TB treatment is commonly determined by the completion of a particular treatment regimen, e.g., a particular drug regimen including, e.g., a number of drug doses ingested over a given period of time. In certain instances TB treatment regimens, including the determined end of TB treatment, are modified according to particular patient characteristics, including, e.g., HIV infection, drug resistance, pregnancy, patient age, etc. In certain instances the end of TB treatment may be determined based on TB assessments described herein in conjunction with the end of a particular treatment regimen, e.g., the end of TB treatment determined by a particular treatment regimen may be altered based on the results of a particular TB assessment. In certain instances the end of the TB treatment may be determined based on a TB assessment described herein independently of any particular treatment regimen, e.g., the end of TB treatment may be determined essentially by one or more TB assessments as described herein. In some instances, such TB assessments useful in determining and/or confirming the end of TB treatment include but are not limited to those assessments described herein end-of-treatment assessments and post-treatment assessments.

In some instances, monitoring of TB treatment includes one or more post treatment assessments or follow-up assessments that are preformed after treatment has been stopped, e.g., to detect a relapse of TB disease or TB infection. The timing and frequency of follow-up assessments will vary and will depend on characteristics of the TB infection (e.g., whether the patient has a latent infection or TB disease), characteristics of the treatment regimen (e.g., the duration of the treatment), characteristics of the subjects medical history (e.g., whether the subject has or has had other lung diseases or treatments), the subject's relative risk of relapse (e.g., whether the subject is immunocompromised, at an increased risk of becoming immunocompromised, or non-immunocompromised), and other considerations (e.g., the availability of the subject for further follow-up testing, the subject's age, quality of life considerations, etc.). In some instances one or more follow-up assessments may be performed in a period after the last treatment ranging from days to years including but not limited to, e.g., from 2 days to 10 years, from 2 days to 5 years, from 2 days to 2 years, from 2 days to 1 year, from 2 days to 9 months, from 2 days to 6 months, from 2 days to 3 months, from 2 days to 2 months, from 2 days to 1 month, from 2 days to 3 weeks, from 2 days to 2 weeks, from 2 days to 1 week, from 1 to 2 weeks, from 1 to 3 weeks, from 1 week to 1 month, from 1 week to 2 months, from 1 to 6 months, from 1 to 5 months, from 1 to 4 months, from 1 to 2 years, from 1 to 3 years, from 1 to 4 years, from 1 to 5 years, from 1 to 6 years, from 1 to 7 years, from 1 to 8 years, from 1 to 9 years, from 1 to 10 years, etc. As discussed above, the frequency of follow-up assessments may vary and in some instances may range, e.g., from frequencies of daily to annually, including but not limited to daily, every other day, every two days, twice weekly, weekly, once every other week, once every three weeks, monthly, once every two months, quarterly, once every four months, once every five months, once every six months, once every seven months, once every eight months, once every nine months, once every ten months, once every eleven months, annually, etc. In some instances, follow-up assessments are performed indefinitely, e.g., for the rest of a subject's life, and the need for such indefinite follow-up may depend on various clinical factors and may be necessary, e.g., due to declining immune function, e.g., due to age related immune system decline.

The present disclosure provides methods for making TB assessments of subjects, such as diagnosing and clinically monitoring TB in a subject, by detecting the levels of one or more biomarkers, including e.g., TB biomarkers, e.g., TB host biomarkers present on the surface of cells obtained from the subject.

In some instances a subject in need of a TB assessment may be a mammal, e.g., a human, suspected of recently having been infected with TB bacteria, e.g., following a TB exposure, e.g., association or contact with a TB infected person or animal or following contact with materials suspected or known to contain TB bacteria, including e.g., TB patient samples or materials known to have been in contact with a TB patient. In some instances, a TB exposure may also include indirect association with a TB infected person, including e.g., occupying a location known to have been previously occupied by a TB infected person or having contact or association with a person known to have had contact or association with a TB infected person. In certain instances an infection or an exposure may be considered recent when the infection or exposure occurs within a time period of less than 1 year from the known or suspected infection or exposure, including but not limited to, e.g., less than 6 months, less than 5 months, less than 4 months, less than 3 months, less than 2 months, less than 1 month, less than 3 weeks, less than 2 weeks, less than 1 week, 1 week, 6 days, 5 days, 4 days, or 3 days.

In some instances a subject in need of a TB assessment may be a person suspected or known to have a latent TB infection or a person suspected or known to have TB disease. A subject infected with TB bacteria may or may not develop TB disease, i.e., a TB infected individual may become symptomatic, developing TB disease or remain asymptomatic for some time thus having a latent TB infection. In TB disease, TB bacteria become active, either in a newly infected individual or an individual with a latent TB infection, when the individual's immune system fails to suppress TB bacterial growth. TB disease may be defined as a TB infection in which TB bacteria are actively multiplying in a host's body. Subjects with TB disease are generally symptomatic and infectious.

A person suspected of or known to have a latent TB infection may be described herein as a latent TB patient or a latent TB infected subject. A latent TB patient may have a latent TB infection for any period of time and the development of TB disease from latent TB depends on the presence or absence of various risk factors. Many people with latent TB infection never develop TB disease. Some people develop TB disease within weeks after becoming infected and others develop TB disease years after latent infection, e.g., after becoming immunocompromised, e.g., from a secondary infection, e.g., from a secondary HIV infection. In immunocompromised people the risk of developing TB disease is much higher than for non-immunocompromised people, i.e., those with normal immune systems. As such, in some instances a subject in need of a TB assessment may be a subject that has recently had an immune compromising event, e.g., a recent infection with an immune compromising agent including e.g., agents that cause immune compromising diseases, e.g., HIV, or recently discovered to be infected with an immune compromising agent.

Based on the cumulative presence or absence of particular risk factors a subject may have a high, normal, or low risk for developing TB disease. Risk factors that increase a subject's chances of developing TB disease, i.e. risk factors that would indicate high risk, include but at not limited to recent infection with TB bacteria, age related weak immune systems (e.g., babies, young children, and elderly individuals), other medical conditions that weaken the immune system (e.g., HIV infection, substance abuse, silicosis, diabetes mellitus, severe kidney disease, low body weight, organ transplants, head and neck cancer, etc.), concurrent medical treatment that weaken the immune system (e.g., immunosuppressive drugs, corticosteroids, anti-rejection drugs following organ transplant, radiation therapy, chemotherapy, treatments for rheumatoid arthritis, treatments for Crohn's disease, etc.).

In some instances, subjects in which a TB assessment is made may or may not have other lung diseases, e.g., another lung in addition to TB or another lung disease in place of TB, e.g., another lung disease that may be mistaken for TB. Such other lung diseases include but are not limited to: Acute Bronchitis, Acute Respiratory Distress Syndrome (ARDS), Asbestosis, Asthma, Bronchiectasis, Bronchiolitis, Bronchiolitis Obliterans Organizing Pneumonia (BOOP), Bronchopulmonary Dysplasia, Byssinosis, Chronic Bronchitis, Coccidioidomycosis (Cocci), COPD, Cryptogenic Organizing Pneumonia (COP), Cystic Fibrosis, Emphysema, Hantavirus Pulmonary Syndrome, Histoplasmosis, Human Metapneumovirus, Hypersensitivity Pneumonitis, Influenza, Lung Cancer, Lymphangiomatosis, Mesothelioma, Middle Eastern Respiratory Syndrome, Nontuberculosis Mycobacterium, Pertussis, Pneumoconiosis (Black Lung Disease), Pneumonia, Primary Ciliary Dyskinesia, Primary Pulmonary Hypertension, Pulmonary Arterial Hypertension, Pulmonary Fibrosis, Pulmonary Vascular Disease, Respiratory Syncytial Virus, Sarcoidosis, Severe Acute Respiratory Syndrome, Silicosis, Sleep Apnea, and the like.

In one embodiment of interest, the expression of surface markers on peripheral blood mononuclear cells (PBMCs) of TB patients from pre-treatment and during standard anti-tuberculosis chemotherapy are measured to identify clinically valuable biomarkers for diagnosis, early treatment response, and cure or absence of cure at the end of treatment. In particular instances, samples from extremely well-characterized TB patients participating in clinical studies, e.g., Bill & Melinda Gates Foundation (BMGF) funded studies, are evaluated using advanced flow cytometric technologies, e.g., those from Becton Dickinson (BD or BDT). In certain instances, methods provided herein are used to discover host candidate biomarkers for TB treatment response and diagnosis or to evaluate previously identified biomarkers for TB treatment response and diagnosis by FACS™ CAP (CAP: combinatorial antibody profile). In some instances host biomarkers are based on PBMC surface molecule expression and are correlated with human TB disease status, extent of disease and treatment outcome, including, e.g., early treatment response and cure at the end of standard anti-TB therapy. Specific examples of such embodiments are provided herein, including e.g., use of BDT developed FACS™ CAP technique to discover peripheral blood cell surface markers which serve as indicators of TB disease status.

BD FACS™ CAP is a multi-dimensional analysis of cell surface proteins for rapid characterization of human cell surface protein expression profiles using semi-automated high-throughput flow cytometry. The technology allows the characterization and quantification of the expression of human cell surface markers using a broad selection of antibodies. The 96-well plate configuration allows for cell testing using more than 200 antibodies against key cell surface markers. The antibodies detect cell surface proteins representing intercellular pathways, apoptosis, cell proliferation, cell-cell signaling, chemotaxis, cell adhesion and cell motility. In other instances, FACS CAP is configured with antibodies for monitoring specific immune functions and the inflammatory response. Several wells of the 96-well plate contain appropriate isotype controls or unstained cells. Antibodies are arrayed randomly 3 by 3 in each well.

One configuration of FACS CAP consists of 229 directly conjugated antibodies arrayed in a 96-well plate as three-color cocktails, which enables the characterization of each of the 229 individual surface markers. Each individual cell type of interest is analyzed on the 96-well screening plates and the data are acquired on a flow cytometer equipped with a high-throughput sampler. The expression level of each marker for each cell type is then calculated using semi-automated custom flow cytometry software. In certain instances, the FACS CAP process of characterizing surface marker profiles in a highly efficient manner is adapted to incorporate automated liquid handling for staining, automated flow cytometry for data acquisition, and standardized algorithms for automated data analysis.

### Compositions

The present disclosure provides compositions useful in practicing the methods disclosed herein for making TB assessments of subjects, such as diagnosing and clinically monitoring TB in a subject by detecting the levels of host TB biomarkers present on the surface of cells obtained from the subject.

In some instances, compositions of the present disclosure include assessment compositions, including e.g., TB monitoring compositions and TB diagnosis compositions. Such compositions include one or more detection reagents that detect aforementioned host TB biomarkers, and in some instances, such detection reagents may be referred to herein as binding members or host TB biomarker binding members. Such binding members may contain a label domain that may be detected by a device, e.g., a flow cytometer, thus allowing qualitative identification or quantification of the level of the host TB biomarker present on a particular event detected by the device, e.g., a cell detected by a flow cytometer. In some instances, such binding members may contain a label binding domain such that the binding member may be detectably labeled by contacting a solution containing the binding member with a detectable label that binds the label binding domain, e.g., contacting a solution containing the binding member with a secondary antibody that is detectably labeled. Any detectably label may be used either in directly or indirectly detectably labeling a binding member of the instant disclosure including those known in the art and those described elsewhere herein.

In some instances, compositions of the instant disclosure may include two or more binding members or host TB biomarker binding members. Such binding members included in compositions of two or more binding members may be detectably labeled such that each class of binding member, e.g., each binding member that binds a particular host TB biomarker, is particularly detectable, i.e. each host TB biomarker detection event is recognizable as to the host TB biomarker bound by a particular binding member. For example, in a composition that includes two binding members that detect two different host TB biomarkers the binding members are detectably labeled with labels that are distinguishable by the detection device. In some instances, binding members included in compositions of two or more binding members may be detectably labeled such that two or more binding members share essentially the same detectable label, e.g., two or more binding members are detectably labeled with labels that cannot be distinguished by the detection device.

In addition, compositions may include one or more additional detectable labels that specifically bind additional cellular markers, e.g., cellular markers that identify an additional characteristic of a cell, e.g., a characteristic other than expression of a particular TB biomarker on the surface of the cell. Detectable labels may bind cellular markers directly or indirectly, i.e. through common binding of a label binding mediator.

### Devices and Systems

Aspects of the invention further include systems for use in practicing the subject methods. Systems of the invention may include a flow cytometry system configured to assay cellular samples (e.g., whole blood, PBMCs, etc.) by measuring signals such as FSC, SSC, ALL, fluorescence emission (e.g., as emission maxima), mass, molecular mass, etc. Steps of the methods described in the previous sections may be performed by the flow cytometry system. Flow cytometers of interest include, but are not limited, to those devices described in U.S. Pat. Nos. 4,704,891; 4,727,029; 4,745,285; 4,867,908; 5,342,790; 5,620,842; 5,627,037; 5,701,012; 5,895,922; 6,287,791; 7,787,197; 8,140,300; and 8,528,427; the disclosures of which are herein incorporated by reference.

In some instances, the flow cytometer includes: a flow channel; a detector module that includes a first detector configured to receive a first signal from the assay region of the flow channel and a second detector configured to receive a second signal from the assay region of the flow channel. The flow cytometer may optionally further include at least a first light source configured to direct light to an assay region of the flow channel (where in some instances the cytometer includes two or more light sources). Optionally further, the flow cytometer may include one or more additional detectors and/or light sources for the detection of one or more additional signals. The one or more additional signals may be produced by one or more additional detectable labels.

The flow cytometer may be configured to produce a data set. The data set may include signal data (e.g., fluorescence excitation and/or emission spectra, fluorescence intensity, fluorescence emission maxima, FSC, SSC, ALL or combinations thereof) for each event in the data set.

The flow cytometry system may also include a “data processing unit”, e.g., any hardware and/or software combination that will perform the functions required of it. For example, any data processing unit herein may be a programmable digital microprocessor such as available in the form of an electronic controller, mainframe, server or personal computer (desktop or portable). Where the data processing unit is programmable, suitable programming can be communicated from a remote location to the data processing unit, or previously saved in a computer program product (such as a portable or fixed computer readable storage medium, whether magnetic, optical or solid state device based).

The flow cytometry system may further include a “memory” that is capable of storing information such that it is accessible and retrievable at a later date by a computer. Any convenient data storage structure may be chosen, based on the means used to access the stored information. In certain aspects, the information may be stored in a “permanent memory” (i.e. memory that is not erased by termination of the electrical supply to a computer or processor) or “non-permanent memory”. Computer hard-drive, CD-ROM, floppy disk, portable flash drive and DVD are all examples of permanent memory. Random Access Memory (RAM) is an example of non-permanent memory. A file in permanent memory may be editable and re-writable.

The memory may store a “module” for execution by the data processing unit, wherein the module is configured to transform the data set from a number transform the data set from a number (X) of signal sets to a number (Y) of marker density sets, wherein Y>X. The marker density sets may include marker expression data (e.g., levels and/or amounts of cellular markers, signals from detectible labels corresponding to cellular markers, etc.) for each cell event in the data set or in a population thereof. The module may be configured to transform the data set based on a categorization of events (e.g. cell events) in the signal set. For example, the same fluorescent signal obtained from two cell events categorized into separate populations may be provided by different detectable labels specific for different cell marker. The module may be configured to distinguish detectable labels (e.g., detectable labels providing a substantially identical signal) based on the categorization.

In certain aspects, the module may be configured to categorize the cell events prior to transforming the data set. Further, the module may be configured to categorize the cell events based on measurements of FSC, SSC, ALL, fluorescence emission or combinations thereof. In other aspects, the cell events may be categorized by an operator (i.e., manually) as described previously.

In addition to the sensor device and signal processing module, e.g., as described above, systems of the invention may include a number of additional components, such as data output devices, e.g., monitors and/or speakers, data input devices, e.g., interface ports, keyboards, etc., fluid handling components, power sources, etc.

In some instances, the systems may further include a cellular sample (e.g., loaded on the flow channel), as prepared according to any of the aspects of the subject methods described above. In certain aspects, the flow cytometer may be a fluorescence activated cell sorter (FACS) instrument or an automated or semi-automated flow cytometer optionally including semi-automated custom flow cytometry software and/or semi- or fully automated liquid handling for staining, semi- or automated flow cytometry for data acquisition, and standardized algorithms for automated data analysis. In certain instances, the device may be a high through put system or include a high through put component.

### Utility

The present disclosure provides methods for the identification of subpopulations of cells collected from subjects suspected of having TB, subjects known to have TB, and patients being treated for TB and the like. Such methods have a number of useful applications described below.

Aspects of the methods described herein include identification of subpopulations of cells expressing host TB biomarkers above or below a particular threshold level useful in obtaining a biomarker signature that can be used in monitoring progression of TB in a subject, e.g., by detecting a first biomarker signature of a blood sample obtained from a subject at a first time point and detecting a second biomarker signature of a blood sample at a second time point and comparing the first and second biomarker signatures to make an assessment of TB progression, wherein the assessment provides for monitoring of the progression of TB from the first time point to the second time point. TB progression may be monitored in TB patients undergoing treatment or patients not undergoing TB, e.g., those patients known to be infected with TB, e.g., those having latent TB, but not undergoing treatment. In certain instances, more than two time points may be utilized in monitoring TB progression.

In certain embodiments, the method of monitoring progression of TB in a subject allows for detecting a pattern of biomarker signatures present in a plurality of samples, e.g., blood samples, obtained from a subject at more than two time points, such as three or more, four or more, five or more, six or more, seven or more, eight or more, nine or more, or ten or more. In general, the time points for detecting a pattern of biomarker signatures can be separated by any amount of time that is desired. For example, the first time point and second time point can be separated by less than 1 week, about 1 week, about 2 weeks, about 3 weeks, about 4 weeks, about 5 weeks, about 6 weeks, about 1 month, about 2 months, about 3 months, about 6 months, or about 1 year or more, such as about 3 or more years.

In general, it will be appreciated by one of skill in the art that the duration of time between the first time point and the second time point must be sufficient to provide for a monitoring of the progression of the TB disease, e.g., the monitoring of TB during TB treatment.

In certain embodiments, the methods of monitoring TB presented herein allow for parallel monitoring of disease progression and disease treatment, e.g., during a treatment regimen for TB. In such embodiments, the method of monitoring TB during treatment will provide information of whether the treatment is improving the condition, or having no effect or an adverse effect on the condition. In such embodiments, the first time point may be either just before, concurrent with, or just after the initiation of a treatment regimen and the second time point may be a time point following a desired treatment period. For example, in such embodiments, the second time point may be about 1 week or more following initiation of treatment, including about 2 weeks, about 3 weeks, about 4 weeks, about 5 weeks, about 6 weeks, about 1 year, about 2 years, or more. For example, the detection of the biomarker signature present in a blood sample obtained from the subject may be determined about once every week or more, including once every two weeks, once every three weeks, once every four weeks, once every five weeks, once every six weeks, once every 2 months, once every 3 months, once every 4 months, once every 5 months, once every 6 months, once every year, once every 2 years, and once every 3 years, to monitor TB progression and efficacy of the treatment regimen.

Certain aspects of the methods, devices, systems and kits presented herein provide for greater efficacy of treatment monitoring and thus great efficacy of treatment as treatments may be tailored to a particular patient's response to treatment. For example, in some instances treatment may be continued longer than intended at the onset of treatment based on a TB assessment performed during treatment that indicates that longer treatment is necessary. In other instances treatment may be discontinued earlier than intended at the onset of treatment based on a TB assessment performed during treatment that indicates that the initially prescribed treatment length is unnecessary.

In certain aspects of the present disclosure, TB assessments are made by comparison of biomarker evaluations or measurements or biomarker signatures to a reference standard. In certain embodiments, methods described herein are useful in deriving such reference standards. In some instances, the reference standard with which a particular subject or patient sample is compared, as described herein, is the patient's or subject's own sample, e.g., the patient's or subject's own sample collected at an earlier time point. In some embodiments, TB monitoring may be performed by making TB assessments, as described herein, by comparison of samples, e.g., patient blood samples or cells of a patient, acquired at different times and/or under different conditions, e.g., at different times during a treatment regimen or under different treatment conditions, e.g., under different treatment regimens or during different phases of treatment.

### Reagents and Kits

Also provided are reagents, devices and kits thereof for practicing one or more of the above-described methods. The subject reagents, devices and kits thereof may vary greatly. Reagents and devices of interest include those mentioned above with respect to the methods of detection of biomarkers and identification of subpopulations of cells expressing biomarkers, e.g., by flow cytometry. The subject kits may include a first detectable label that specifically binds to a first cellular marker and a second detectable label that specifically binds to a second biomarker. The first and second detectable labels may provide a substantially identical signal or substantially different signals. A detectable label may include a label domain and a binding member specific for a biomarker, as described in the previous section.

Kits useful for practicing one or more of the above-described methods may include one or more of such reagents and devices including e.g., reagents and devices for biomarker detection, reagents and devices for identification of subpopulations of cells expressing one or more biomarkers, reagents and devices for collecting, storing, preparing, processing, samples prior or during execution of any of the methods described herein, and devices for interpreting, storing, converting, displaying, or disseminating data pertaining to assessments made according to the methods described herein. In addition, the kits may include one or more calibration or reference reagents, e.g., for use in calibration of a device, including e.g., a flow cytometer, or for configuration of a device, including e.g., configuration of a flow cytometer, including e.g., configuration of threshold values, e.g., biomarker threshold values, to be used in assessments as described herein. In addition, the kit may include one or more additional compositions that are employed, including but not limited to: buffers, diluents, cell lysing agents, etc., which may be employed in a given assay. The above components may be present in separate containers or one or more components may be combined into a single container, e.g., a glass or plastic vial.

In addition, the kit may include one or more additional detectable labels that specifically bind additional cellular markers, e.g., cellular markers that identify an additional characteristic of a cell, e.g., a characteristic other than expression of a particular TB biomarker on the surface of the cell. Detectable labels may bind cellular markers directly or indirectly, i.e. through common binding of a label binding mediator. Detectable labels may be provided in separate containers or mixed in the same container.

The kit may also include one or more cell fixing reagents such as paraformaldehyde, glutaraldehyde, methanol, acetone, formalin, or any combinations or buffers thereof. Further, the kit may include a cell permeabilizing reagent, such as methanol, acetone or a detergent (e.g., triton, NP-40, saponin, tween 20, digitonin, leucoperm, or any combinations or buffers thereof. Other protein transport inhibitors, cell fixing reagents and cell permeabilizing reagents familiar to the skilled artisan are within the scope of the subject kits.

The kit may further include reagents for performing a flow cytometric assay. Examples of said reagents include buffers for at least one of reconstitution and dilution of the first and second detectable molecules, buffers for contacting a cell sample with one or both of the first and second detectable molecules, wash buffers, control cells, control beads, fluorescent beads for flow cytometer calibration and combinations thereof.

The detectable labels and/or reagents described above may be provided in liquid or dry (e.g., lyophilized) form. Any of the above components (detectable labels and/or reagents) may be present in separate containers (e.g., separate tubes, bottles, or wells in a multi-well strip or plate). In addition, one or more components may be combined into a single container, e.g., a glass or plastic vial, tube or bottle.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, removable drive, flash drive, etc., on which the information has been recorded. Yet another means that may be present is a website address which may be used via the internet to access the information at a removed site. Any convenient means may be present in the kits.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, and kits for genetic manipulation referred to in this disclosure are available from commercial vendors such as BioRad, Stratagene, Invitrogen, Sigma-Aldrich, and ClonTech.

### Example 1

### Materials and Methods

Newly diagnosed TB patients, healthy individuals and patients with other lung diseases (OLD) were recruited into the study after obtaining informed consent. Diagnosis of TB was made based on medical history, physical examination and the detection of TB by smear microscopy. HIV status or other health conditions (other than TB) of participants were recorded. Blood from each patient was collected before the start of therapy (T0), after 4 weeks of TB therapy (W4) and at the end of therapy (W24). Blood from control subjects was mostly collected once and from few subjects, four weeks after the first collection. All samples were transported to the TB immunology laboratory where they were processed and where PBMCs were prepared from blood samples by Ficoll centrifugation and stained in FACS CAP plates according to written protocols. For each patient, duplicate plates were prepared to account for problems that may occur in plates during cell staining or acquisition on the cytometer.

Data acquisition of FACS CAP plates was performed on the high through put system (plate reader) of the BD FACS Calibur. 30,000 events were collected from each well for the duplicate plates.

### FACS Analysis

All FACS analysis was performed manually by using FlowJo software and by gating on the lymphocyte population. The cut off for the positive population was determined by comparison to the isotype controls. In some instances, where the isotype control did not appear reliable, an adjustment was made by relying on the profile of expression in dot plots using parameters two by two (FSC, SSC, FL1, FL2, FL3). When the decision on the expression for given markers was not clear, the markers were “flagged” and the decision was made for those markers to be further investigated at a later point. All patients were analyzed but only 33 patients for which samples from 3 time points (T0, W4 and W24) were available were considered for final statistical analysis.

### Statistical Analysis

Repeated measures ANOVA

Repeated measures analysis deals with response outcomes measured on the same experimental unit at different times or under different conditions. Longitudinal data are a common form of repeated measures in which measurements are recorded on individual subjects over a period of time. In this study, repeated measures ANOVA is appropriate to test for the difference in the mean expressions of biomarkers collected from the same 33 subjects but at different time points (baseline, week 4 and week 24).

The univariate repeated measures ANOVA model is defined as follows:

yᵢⱼ=μ+πᵢ+τⱼ+eᵢⱼ, for i=1, . . . ,33;j=1,2,3.

μ is the grand mean, πᵢ is the random effect due to individual difference component for subject (constant over time), τⱼ is the effect of time, and eᵢⱼ is the error for subject i and time j. In order to avoid over-parameterization, we let τⱼ₌₁³τⱼ=0.

The assumptions for this model are:

πᵢ˜N (0,σ_{π}²), which indicates the random effect due to subjects that are assumed to be normally distributed with zero mean and constant variance at σₑ².

eᵢⱼ˜N (0,σₑ²), which indicates the random errors that are assumed to be normally distributed with zero mean and constant variance at σ_{π}².

The repeated measures ANOVA is used to test the null hypothesis: H₀: τ₁=τ₂=τ₃=0, which indicates that the means of three time points (baseline, week 4 and week 24) are all equal, i.e., μⱼ=μ+τ₁ are all equal for j=1,2,3. Alternatively, any Tⱼ≠0 for j=1,2,3, in other words, at least the means from two time points are different.

Since this study is comparing 252 biomarkers simultaneously, effects of multiple comparisons should be considered. Bonferroni correction is a procedure that has been widely used in multiple comparisons. Based on the Bonferroni correction, if the size of the test α=0.05, only biomarkers with

$$p \text{-} value \leq \frac{0.05}{252} \approx 0.0002$$

should be considered significant.
Paired t-Tests

Once differences are detected from the previous repeated measures ANOVA tests, we used paired t-tests to evaluate whether the means are different at any two given time points, e.g., baseline (T0) vs. week 4 (W4), baseline (T0) vs. week 24 (W24), or week 4 (W4) vs. week 24 (W24). Paired t-tests are a form of blocking, and therefore have greater power than unpaired t tests when the paired units are similar with respect to the noise factors in the two groups being compared. The null hypothesis under testing will be: μ₁=μ₂, i.e., equal means from any paired groups.

### Independent Two-Sample t Tests

Since the healthy controls, TB patients, and patients with other lung diseases are groups of different independent subjects with unequal sample sizes, the independent two-sample t test was used to evaluate whether the means are different between any two groups, e.g., TB patients at baseline (T0) vs. healthy, TB patients at week 24 (W24) vs. healthy, TB patients at baseline (T0) vs. other lung diseases, or healthy vs. patients with other lung diseases. The Welch's t-test was chosen for this study with Welch (or Satterthwaite) approximation to the degrees of freedom in the tests. The null hypothesis under testing will be: μ₁=μ₂, i.e., equal means from any two independent groups.

### Results

Repeated Measure ANOVA test Results

Null hypothesis: μ_{T0}=μ_{W4}=μ_{W24}

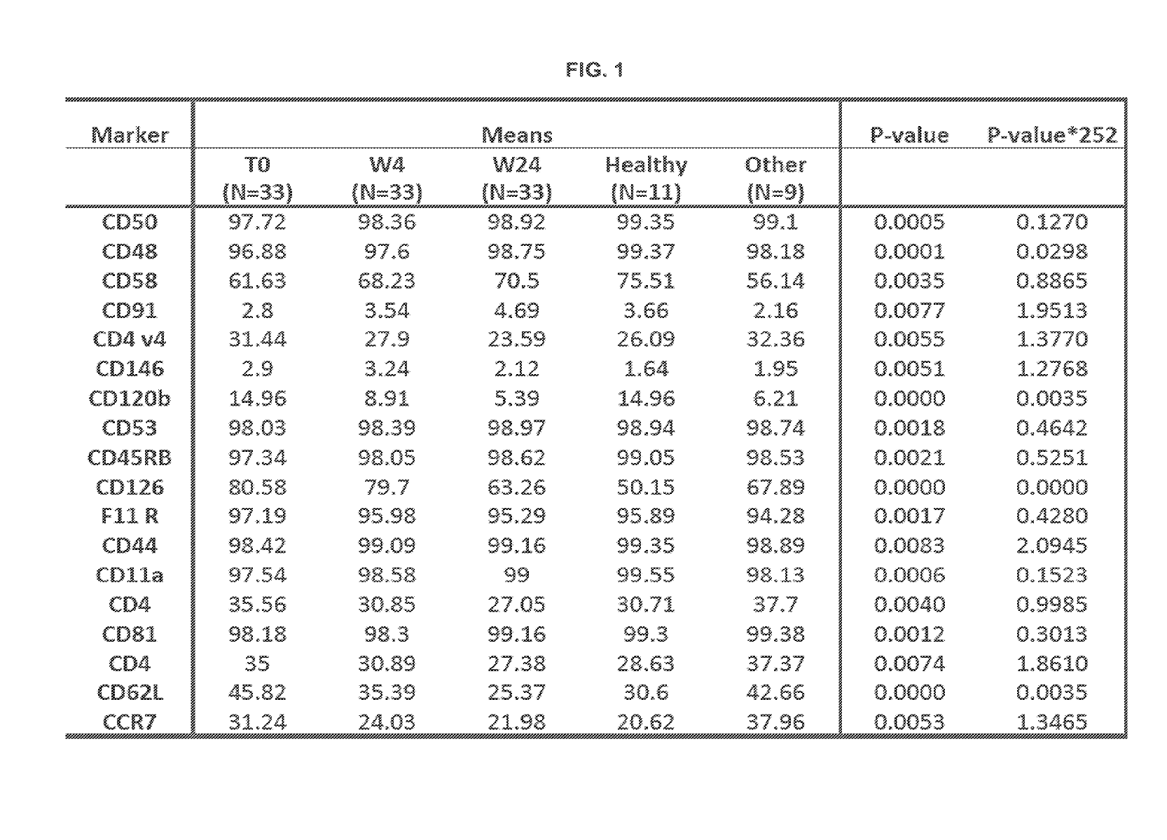

Table 1, provided in FIG. 1, shows a selection of 29 markers with p-value<0.01 (the p-value threshold of 0.01 allows to examine a large number of markers with change or trend). After the Bonferroni correction (see above) was applied, few markers showed

$$p \text{-} value < 0.0002 ( \leq \frac{0.05}{252} \approx 0.0002 ,$$

where 252 is the number of markers we interrogated in the study). Biomarkers in Table 1 with p-values less than 0.05 after Bonferroni correction are in bold. As shown in Table 1, the expression of the markers CD120b, CD126 and CD62L decreased significantly during the course of therapy and especially at the end of therapy. Even though the p-values for markers such as CD29 and CD48 are also significant, there is not a biologically meaningful significance as change in expression is small and more than 95% of the cells were positive at all time points.

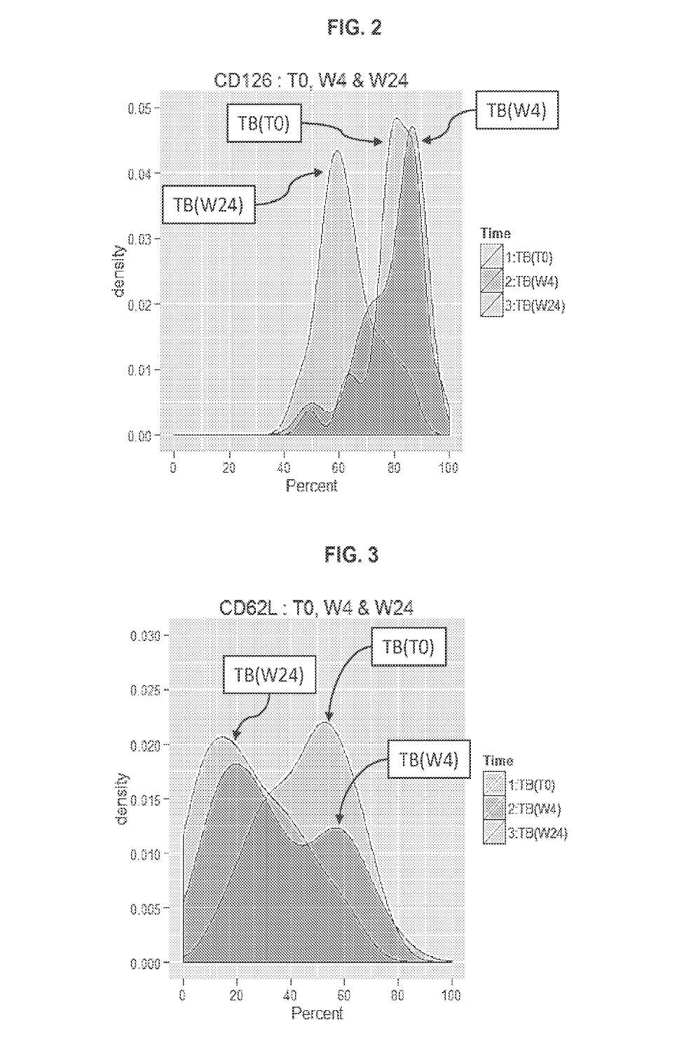

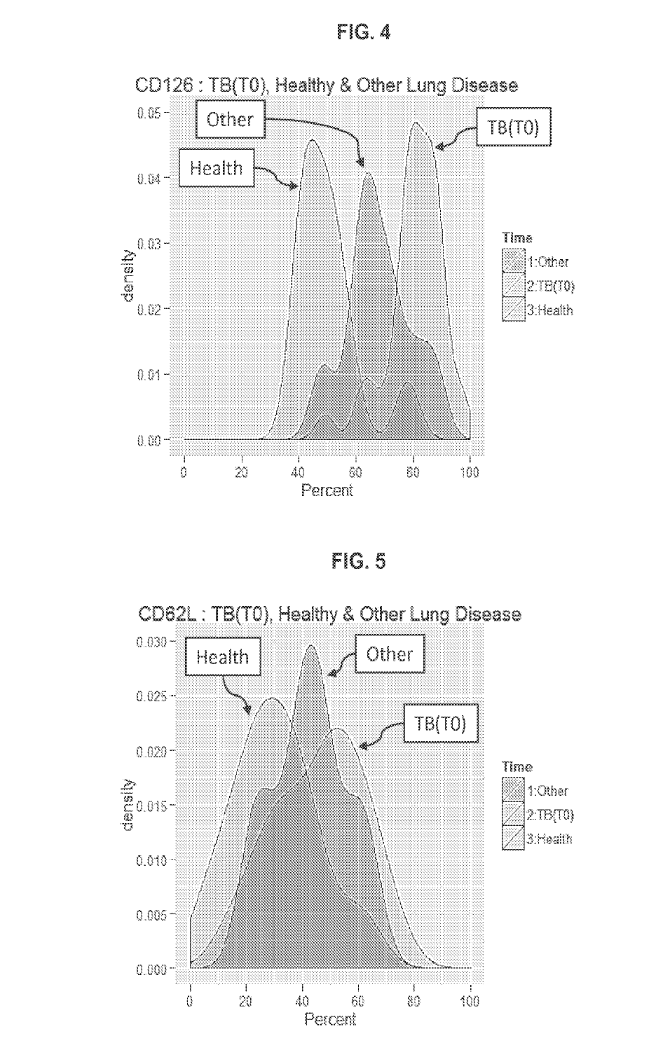

The density curves provided in FIG. 2-5, illustrate the distribution of the expression of CD126 and CD62L among all patients over time and among healthy patients and patients with OLD. FIG. 2-5 display Kernel density curves for CD126 and CD62L from different groups, including the distribution of expression of CD126 and CD62L in TB patients before therapy (T0), week 4 and week 24 (FIG. 2 and FIG. 3) and the distribution of expression of CD126 and CD62L in TB patients at T0, healthy subjects and patients with other lung disease (FIG. 4 and FIG. 5). The graphs show clearly that the expression of CD126 and CD62L is low at week 24 as compared to T0 or week 4 (FIG. 2 and FIG. 3). The curves also show that the distribution of the expression at week 24 is similar to the distribution seen in healthy controls and is different from TB patients or patients with other lung diseases.

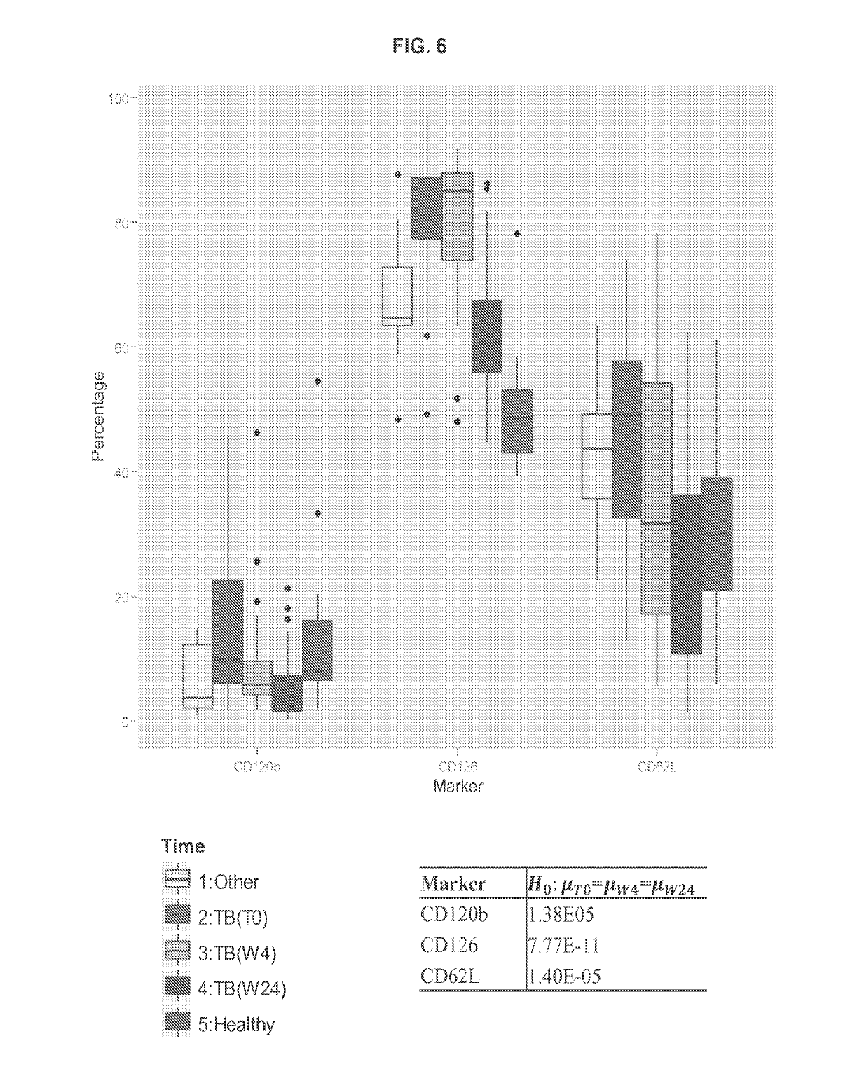

The average expression of CD126, CD120b and CD62L in the 33 TB patients at T0, week 4 and W24 as well as in control groups (Healthy and patients with OLD) is displayed in FIG. 6. It is clear that the expression of CD126, CD120b and CD62L (and especially CD126 and CD62L) in TB patients is higher than in healthy controls and decreases at week 24 to a level close to healthy controls.

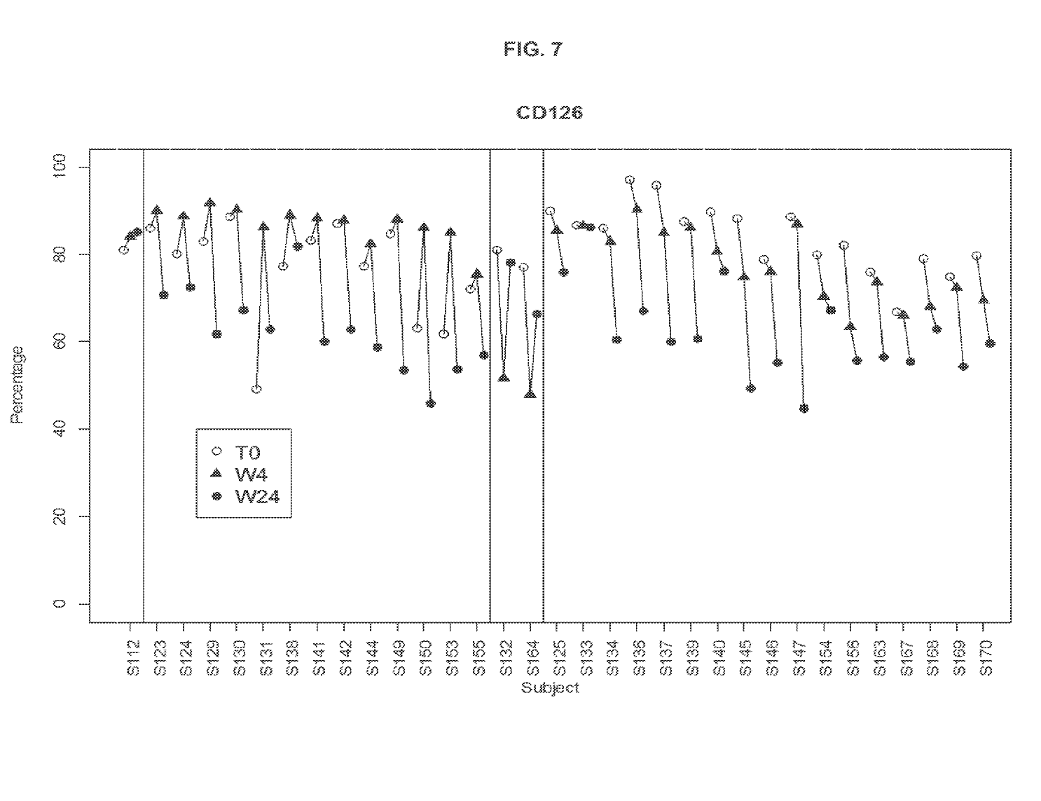

The expression of CD126 was individually examined in 33 patients (FIG. 7), it was clear that the level of expression of this marker was consistently lower at the end of TB therapy (W24) for all patients (filled circles). This analysis further revealed that patients were split into two groups. For one group (FIG. 7, left), the CD126 expression was up regulated at week 4 (triangles), as compared to the expression at T0 (open circles), before going down at week 24. For the second group; the expression of CD126 was down regulated at week 4 (triangles), as compared to the expression at T0 (open circles), and even more down regulated at week 24 (FIG. 7, right).

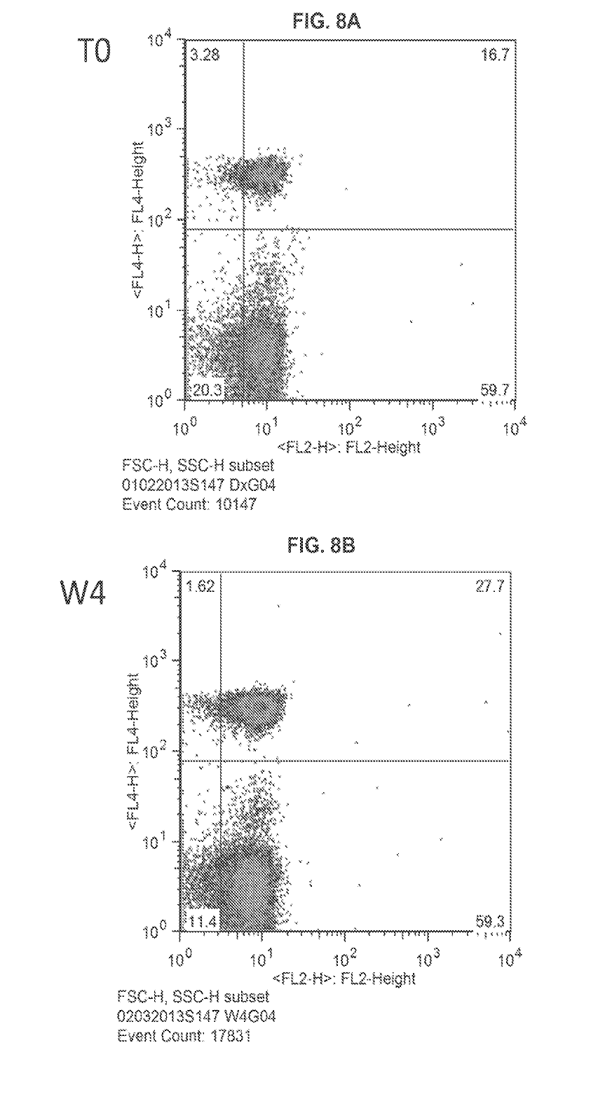

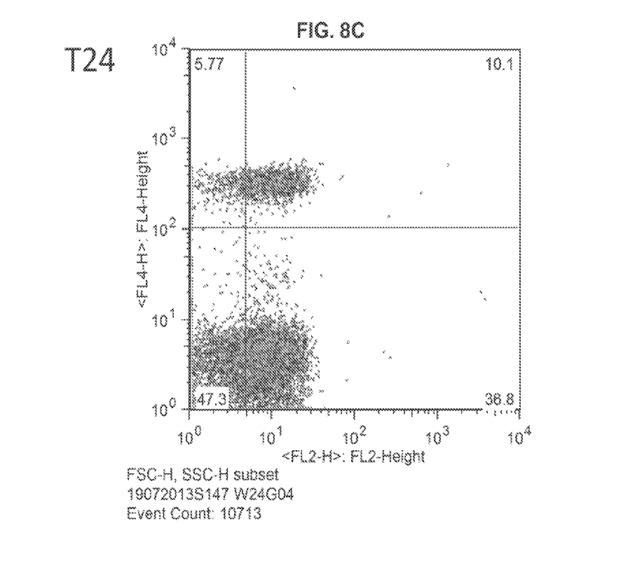

Examination of co-expression of CD4 and CD126 on patient cells was performed by labeling with both anti-CD4 and anti-CD126 antibodies within the same sample well. This analysis is presented for an exemplary patient (S147) at three time points (T0, W4, and W24) in FIG. 8A-C showing expression of CD126 on the X-axis and expression of CD4 on the Y-axis. Both CD4 positive and CD4 negative populations expressed the CD126 surface marker; however, as illustrated in FIG. 8A-C, the down regulation of CD126 occurs primarily in the CD4 negative population.

Paired t-Test Results

Null hypothesis: μ_{T0}=μ_{W4}

Null hypothesis: μ_{T0}=μ_{W24}

Null hypothesis: μ_{W4}=μ_{W24}

Paired t-tests were applied to determine markers that would distinguish between two time points. The pre-therapy (T0) time point was compared with week 4 or week 24 (T0 vs W4 and T0 vs W24) and patients at week 4 were compared with patients after therapy at week 24 (W4 vs W24). A set of markers with p-value<0.01 was selected for each comparison and the Bonferroni correction was applied.

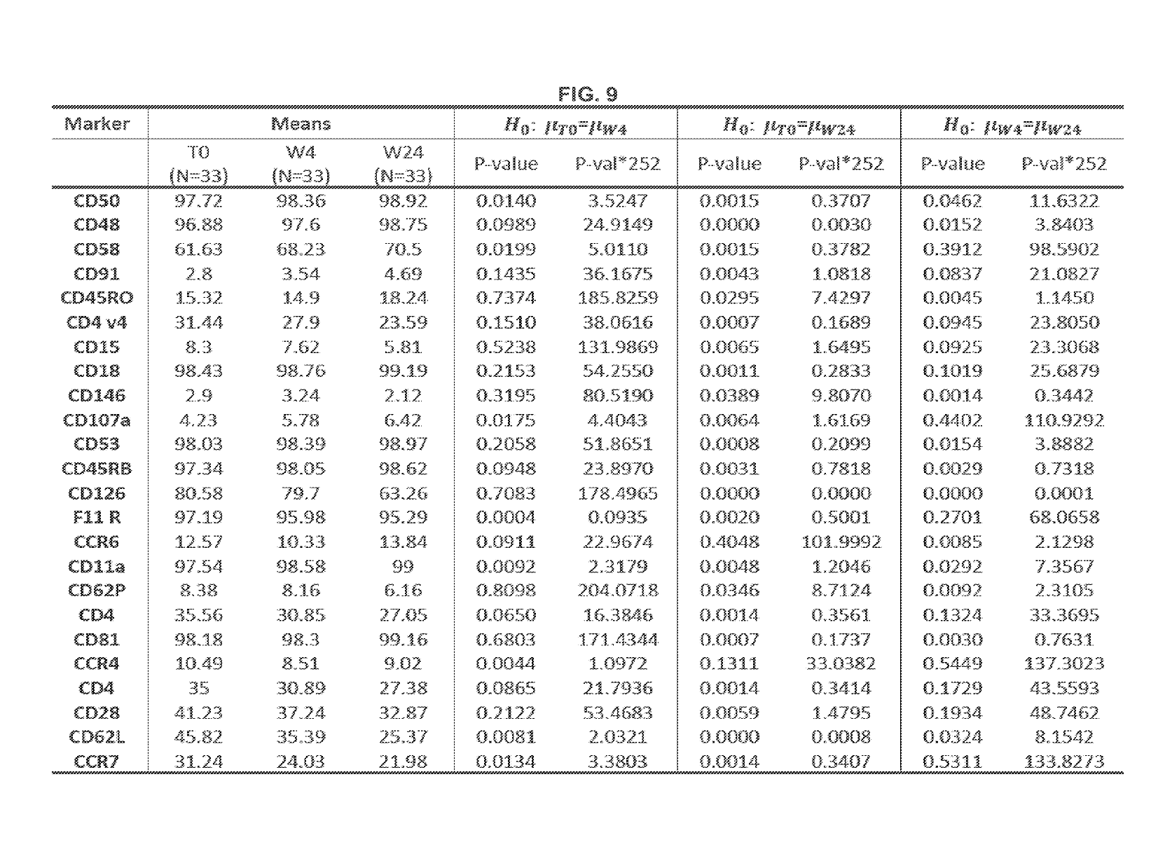

Table 2, provided in FIG. 9, provides the results of this analysis, displaying a selection of biomarkers with p-values less than 0.01 and markers with p-values less than 0.05 after the Bonferroni correction in bold. As shown in Table 2, CD120b, CD126 and CD62L are markers for which the change in expression is significant for this test. Also apparent is that the difference in expression is more prominent when comparing between patients before treatment (T0) and at the end of the treatment (W24) than when comparing between T0 and W4.

A higher number of markers, p-values italicized in Table 2, show trends for changes but not statistical significance after of the stringent Bonferroni correction. For example, markers such as CD58, CD11a and CD4 distinguish between TB patients at the start of therapy and the end of therapy.

Independent Two-Sample t-Test Results

Null hypothesis: μ_{T0}=μ_{Healthy}

Null hypothesis: μ_{T0}=μ_{Other lung disease}

Null hypothesis: μ_{Healthy}=μ_{Other lung disease}

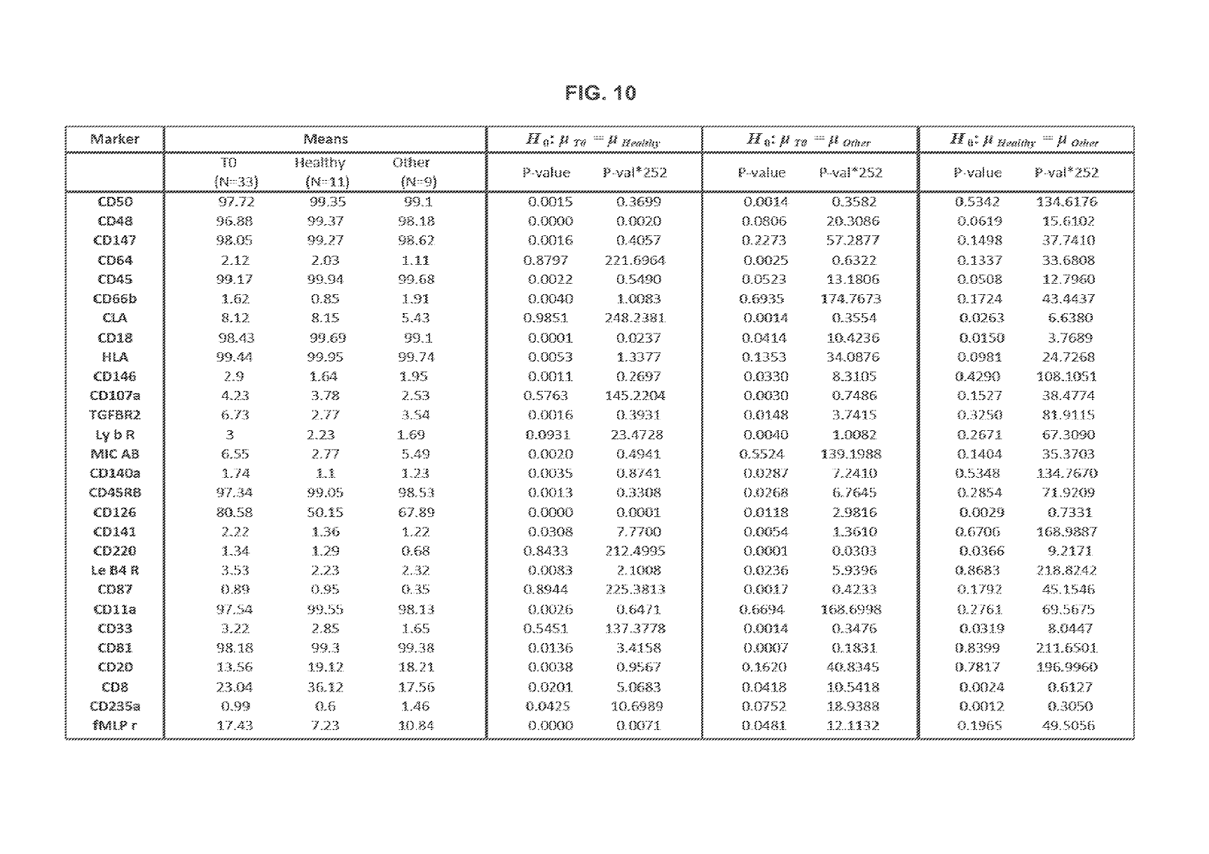

An independent two-sample t-test was performed on the data generated by the flow Jo analysis in order to compare between TB patients before therapy and healthy controls or patients with other lung disease and between healthy controls and patients with other lung diseases. Table 3, provided in FIG. 10, provides the results of this analysis, displaying a selection of biomarkers with p-values less than 0.01 and markers with p-values less than 0.05 after the Bonferroni correction in bold.

As shown in Table 3, CD126 is a marker that distinguishes significantly between TB patients and healthy controls. Also shown in Table 3, fMLP r (fMLP receptor) was shown to distinguish significantly between TB patients and healthy controls.

Even though not significant after the Bonforroni correction, groups of other markers, p-values in Table 3, distinguish between TB patients and patients with other lung diseases or between healthy controls and patients with other lung diseases. Also, as seen in the data, the difference between TB patients before therapy and healthy controls is more prominent than the difference between TB patients and patients with OLD or between healthy controls and patients with OLD.

### Other Results

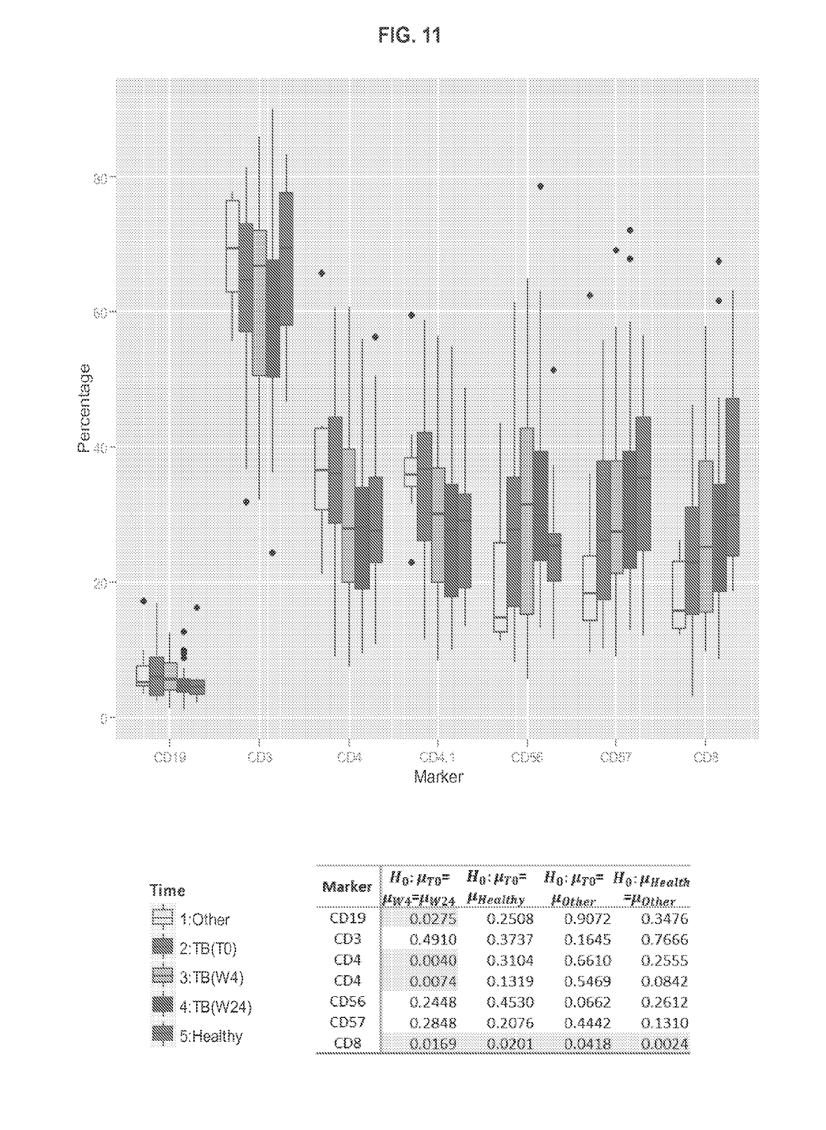

Additional sets of markers show a trend in expression when comparisons are performed between groups or time points. Exemplary trends are depicted in FIG. 11 which displays a representation of marker for which the expression changes during the course of therapy and for which p-values are low but not significant after the application of the Bonferroni correction. For example, FIG. 11 shows that the expression of CD4 positive cells is higher in TB patients before therapy and tends to decrease after the start of therapy to reach a level comparable to healthy controls. FIG. 11 also shows that population expression the CD8 or CD57 increase after the start of therapy.

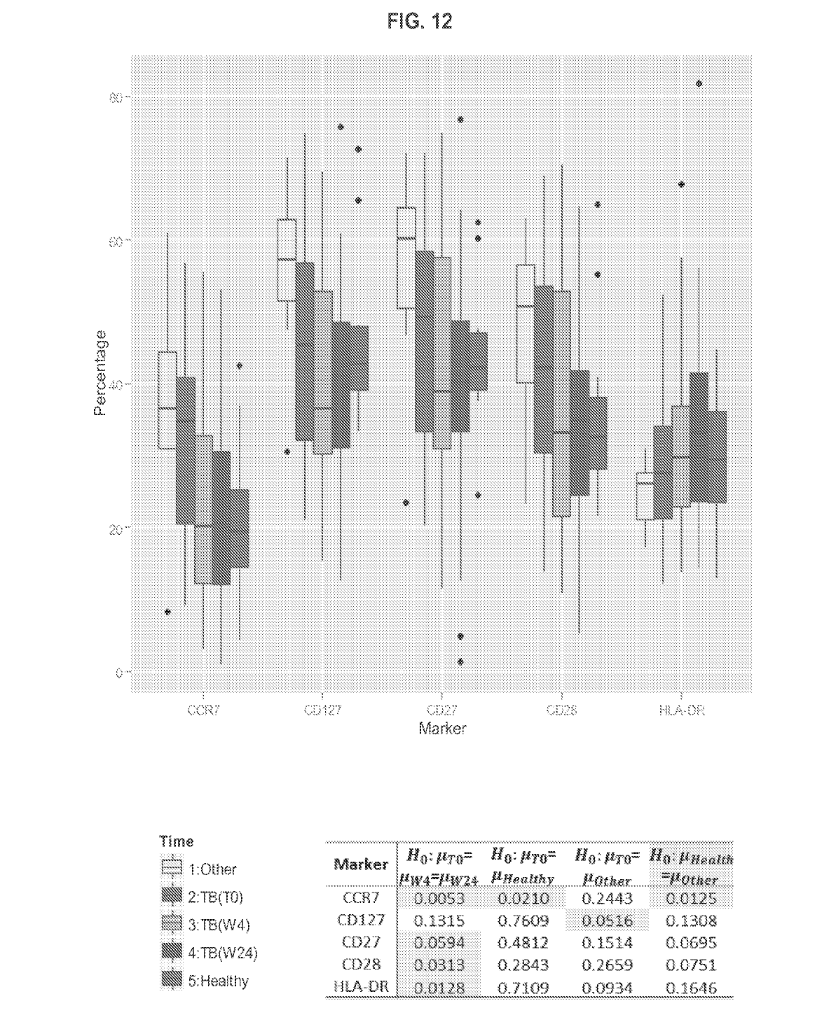

Similarly, other markers of biological significance such as CCR7, CD127, CD27 and HLA-DR, show consistent trends (increase or decrease) in the change of expression of the markers over therapy time points and/or in comparison the control groups but without statistical significance following the Bonferroni correction. Examples of such consistent trends are provided in FIG. 12 which displays a representation of biologically significant markers for which the expression changes during the course of therapy and p-values are low but not significant after application of the Bonferroni correction.

In addition, five TB patients were recruited to determine the change in surface marker expression levels after antigen stimulation. Samples collected from five actively infected TB patients were used to determine if there was a change in expression of surface markers of purified protein derivative (PPD) stimulated PBMC's. Upon receiving the blood, PBMC isolation was performed. Once the protocol reached the second wash step, the samples were split in half. The protocol was continued with one half of the cells while the other half was re-suspended in media at a concentration of 1×10⁶ cells/mL. The stimulant of choice was PPD at a concentration of 10 μg/mL. The cells were incubated overnight at 37° C., 5% CO₂. The following morning the cells were washed twice in PBS and the protocol was continued, data was obtained as described and statistical analysis was performed.

Markers such as CD41a, CD45Ra and CD61 were down-regulated when comparing stimulated with unstimulated PBMC's and expression for markers such as CD4v4, CD49a and CD62L were up-regulated in stimulated compared with unstimulated PBMC's (Table 7, below) although it was noted that these changes did not reach significance.

Comparison of baseline biomarker levels were further stratified based on patient outcome characteristics and statistical analysis was performed. Patient outcomes were clinically defined as “definitive cure”, “probable cure” and “no cure” or were based on PET scan or combined PET-CT scan. PET scan outcomes were defined as “good” or “poor”. Combined PET-CT scans, performed at end of treatment, were defined as “improved” or “mixed”. Baseline biomarker expression levels stratified by outcome were compared by ANOVA and further statistical analysis was performed by individual t-tests (p<0.008) and non-parametric testing (Mann-Whitney-U test). CD18, CD11a, CD50, CD48 and CD53 were all found to statistically differentiate baseline biomarker levels between patients with “definitive cure” and “not cured” outcomes. CD45RO and CD4v4 were all found to statistically differentiate baseline biomarker levels between patients with “mixed” and “improved” outcomes as determined by PET scan. CD18, CD11a, CD62P and CD81 were all found to statistically differentiate baseline biomarker levels between patients with “good” and “poor” outcomes as determined by combined PET-CT scan.

TABLE 7

| | Patient S191 | | Patient S192 | | Patient S193 | | Patient S201 | | Patient S203 | | |
|---|---|---|---|---|---|---|---|---|---|---|---|
| Marker | Stim | Unstim | Stim | Unstim | Stim | Unstim | Stim | Unstim | Stim | Unstim | P-value |
| CD41a | 5.04 | 30.9 | 11.6 | 37.1 | 2.09 | 15.3 | 7.64 | 24.3 | 12.9 | 12.1 | 0.0302 |
| CD45RA | 64.9 | 74.9 | 58.2 | 57 | 0.71 | 57.8 | 34.3 | 39.8 | 72.9 | 87.9 | 0.1689 |
| CD61 | 7.14 | 14.5 | 8.34 | 29.8 | 3.52 | 13 | 2.47 | 12.8 | 12.1 | 11.5 | 0.0532 |
| CD4 v4 | 37 | 28.5 | 44.2 | 25.3 | 42.5 | 37.1 | 62.5 | 55.7 | 9.82 | 7.37 | 0.04 |
| CD49a | 32.1 | 16.1 | 3.95 | 2.49 | 9.58 | 1.9 | 15.7 | 5.13 | 9.38 | 4.34 | 0.03 |
| CD62L | 59.2 | 37.6 | 60.3 | 39 | 36 | 31.4 | 77.4 | 60.5 | 24.9 | 19.5 | 0.0205 |

### DISCUSSION

CD126, CD62L and CD120b are significantly down regulated during the course of therapy especially when comparing between T0 and end of therapy. CD126 is significantly decreased at week 24 after therapy and also significantly distinguishes between TB patients and healthy controls; such a marker profile is consistent with markers or a marker profile for therapy efficacy and TB diagnostics. Other markers such as CD4, CD8, CD56 and CCR7 show a trend of either a decrease or increase during the course of therapy. Even though the highly conservative statistical methods used, e.g., the Bonferroni correction, resulted in differences that were not statistically significant following statistical analysis, markers showing trends of either a decrease or increase during the course of therapy, e.g., those seen for CD4, CD8, CD56, CCR7, are of high biological significance. Markers such as CD58, CD11a and CD4 have diagnostic value as they distinguish, e.g., between TB patients before the start of therapy and the end of therapy.

Notwithstanding the appended clauses, the disclosure set forth herein is also defined by the following clauses:

1. A method of obtaining a tuberculosis assessment for a subject, the method comprising:

identifying a subpopulation of a cellular sample of the subject having an expression level for a tuberculosis host biomarker below a threshold expression level to produce a biomarker signature for the cellular sample, wherein the tuberculosis host biomarker is selected from the group consisting of CD120b, CD126 and CD62L and combinations thereof; and

obtaining a tuberculosis assessment for the subject from the biomarker signature.

2. The method of Clause 1, further comprising identifying a subpopulation of the cellular sample having an expression level below a threshold expression level of one or more additional tuberculosis host biomarkers selected from CD4, CD8, CD56, CD57 and CCR7 and combinations thereof.
3. The method of Clause 1, wherein the tuberculosis assessment is a treatment assessment.
4. The method of Clause 3, further comprising identifying a subpopulation of the cellular sample having an expression level above a threshold expression level for CD58.
5. The method of Clause 1, wherein the tuberculosis assessment is a diagnosis.
6. The method of Clause 5, further comprising identifying a subpopulation of the cellular sample having an expression level below a threshold expression level of one or more tuberculosis host biomarkers selected from the group consisting of CD8 and CD57 and combinations thereof.
7. The method of Clause 5 or 6, further comprising identifying a subpopulation of the cellular sample having an expression level below a threshold expression level of fMLP r.
8. The method of Clause 5 or 6, wherein the diagnosis comprises obtaining a tuberculosis diagnosis for a non-tuberculosis lung disease.
9. The method of Clause 1, wherein the tuberculosis assessment comprises a prediction of the likelihood of a positive treatment outcome or a negative treatment outcome.
10. The method of Clause 9, further comprising identifying a subpopulation of the cellular sample having an expression level below a threshold expression level of one or more tuberculosis host biomarkers selected from the group consisting of CD18, CD11a, CD50, CD48, CD53, CD62P, CD81, CD45RO, and CD4v4 and combinations thereof.
11. The method of any of Clauses 1 to 10, wherein the cellular sample is a blood sample.
12. The method of Clause 11, wherein the subpopulation of the cellular sample comprises peripheral blood mononuclear cells.
13. The method of Clause 12, wherein the method further comprises identifying the peripheral blood mononuclear cells using a CD4 based peripheral blood mononuclear cell identification protocol.
14. The method of Clause 11, wherein the method further comprises subjecting the subject to antigen stimulation prior to collection of the blood sample.
15. The method of Clause 11, wherein the method further comprises subjecting the sample to antigen stimulation prior to analysis.
16. The method of Clause 14 or 15, wherein the antigen stimulation comprises Mycobacterium tuberculosis antigen stimulation.
17. The method of any of Clauses 1 to 16, wherein the identifying comprises flow cytometry.
18. A tuberculosis assessment composition, the composition comprising:

a collection of two or more detectably labeled tuberculosis host biomarker specific binding members, wherein the tuberculosis host biomarkers are selected from the group consisting of CD120b, CD126, CD62L, fMLP r and combinations thereof.

19. The composition of Clause 18, further comprising an additional detectably labeled specific binding member that specifically binds to an additional tuberculosis host biomarker selected from the group consisting of CD4, CD8 and CD57.
20. The composition of Clause 18, further comprising an additional detectably labeled specific binding member that specifically binds to an additional tuberculosis host biomarker selected from the group consisting of CD18, CD11a, CD50, CD48, CD53, CD62P, CD81, CD45RO, and CD4v4.
21. A kit comprising:

a collection of two or more detectably labeled tuberculosis host biomarker specific binding members, wherein the tuberculosis host biomarkers are selected from the group consisting of CD120b, CD126, CD62L, fMLP r and combinations thereof.

22. The kit of Clause 21, further comprising an additional detectably labeled specific binding member that specifically binds to an additional tuberculosis host biomarker selected from the group consisting of CD4, CD8, and CD57.
23. The kit of Clause 21, further comprising an additional detectably labeled specific binding member that specifically binds to an additional tuberculosis host biomarker selected from the group consisting of CD18, CD11a, CD50, CD48, CD53, CD62P, CD81, CD45RO, and CD4v4.
24. A flow cytometry system comprising:

a flow cytometer comprising a flow cell;

a light source configured to direct light to an assay region of the flow cell;

a first detector configured to receive light of a first emission wavelength emitted by a first detectably labeled tuberculosis host biomarker specific binding member present in a cellular sample in the assay region; and

a signal processing module configured to receive signals from the first detector and output a result of whether a subpopulation of cells bound to the first detectably labeled tuberculosis host biomarker specific binding member is present in the cellular sample.

25. The flow cytometry system of Clause 24, wherein the flow cell further comprises a cellular sample comprising a first detectably labeled tuberculosis host biomarker specific binding member.
26. The flow cytometry system of Clauses 24 or 25, wherein the tuberculosis host biomarker is selected from the group consisting of CD120b, CD126, and CD62L and combinations thereof.
27. The flow cytometry system of Clause 24, further comprising:

a second detector configured to receive light of a second emission wavelength emitted by a second detectably labeled tuberculosis host biomarker specific binding member, wherein the signal processing module is configured to receive signals from the first and the second detectors and output a result of whether a subpopulation of cells bound to the first, the second, or both the first and the second detectably labeled tuberculosis host biomarker specific binding members is present in the cellular sample.

28. The flow cytometry system of Clause 27, wherein the flow cell further comprises a cellular sample of a subject; a first detectably labeled tuberculosis host biomarker specific binding member; and a second detectably labeled tuberculosis host biomarker specific binding.
29. The flow cytometry system of Clauses 27 or 28, wherein the tuberculosis host biomarkers are selected from the group consisting of CD120b, CD126, and CD62L and combinations thereof.
30. The flow cytometry system of Clauses 27 or 28, wherein the tuberculosis host biomarker of the first detectably labeled tuberculosis host biomarker specific binding member is selected from the group consisting of CD120b, CD126, and CD62L and the tuberculosis host biomarker of the second detectably labeled tuberculosis host biomarker specific binding member is selected from the group consisting of CD4, CD8, CD57, CD58, CCR7, and fMLP r.
31. The flow cytometry system of Clauses 27 or 28, wherein the tuberculosis host biomarkers are selected from the group consisting of CD18, CD11a, CD50, CD48, CD53, CD62P, CD81, CD45RO, and CD4v4 and combinations thereof.
32. The flow cytometry system of Clauses 27 or 28, wherein the tuberculosis host biomarker of the first detectably labeled tuberculosis host biomarker specific binding member is selected from the group consisting of CD120b, CD126, and CD62L and the tuberculosis host biomarker of the second detectably labeled tuberculosis host biomarker specific binding member is selected from the group consisting of CD18, CD11a, CD50, CD48, CD53, CD62P, CD81, CD45RO, and CD4v4.
33. The flow cytometry system of Clauses 27 or 28, wherein the signal processing module is configured to output a result of whether two or more subpopulations of cells bound to the detectably labeled tuberculosis host biomarker specific binding members are present in the cellular sample.
34. The flow cytometry system of Clauses 24 or 27, further comprising an additional detector configured to receive light scattered by cells present in the cellular sample.
35. The flow cytometry system of Clauses 25 or 28, wherein the cellular sample comprises human cells.
36. The flow cytometry system according Clauses 24 or 27, wherein the system is configured to:

identify from the result a subpopulation of a cellular sample of a subject having an expression level for a tuberculosis host biomarker below a threshold expression level to produce a biomarker signature for the cellular sample, wherein the tuberculosis host biomarker is selected from the group consisting of CD120b, CD126 and CD62L and combinations thereof; and

obtain a tuberculosis assessment for the subject from the biomarker signature.

37. A computer readable medium comprising programming for execution by a computer, comprising:

instructions for analyzing signals produced by a detector configured to receive light of a emission wavelength emitted by a detectably labeled tuberculosis host biomarker specific binding member to produce data;

instructions for storing the data on a computer readable medium; and

instructions for outputting the data.

38. The computer readable medium according to Clause 37, wherein the programming further comprises instructions for:

identifying from the data a subpopulation of a cellular sample of a subject having an expression level for a tuberculosis host biomarker below a threshold expression level to produce a biomarker signature for the cellular sample, wherein the tuberculosis host biomarker is selected from the group consisting of CD120b, CD126 and CD62L and combinations thereof; and

obtaining a tuberculosis assessment for the subject from the biomarker signature.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

That which is claimed is:

1. A method of obtaining a tuberculosis assessment for a subject, the method comprising:
identifying a subpopulation of a cellular sample of the subject having an expression level for a tuberculosis host biomarker below a threshold expression level to produce a biomarker signature for the cellular sample, wherein the tuberculosis host biomarker is selected from the group consisting of CD120b, CD126 and CD62L and combinations thereof; and
obtaining a tuberculosis assessment for the subject from the biomarker signature.

2. The method of claim 1, further comprising identifying a subpopulation of the cellular sample having an expression level below a threshold expression level of one or more additional tuberculosis host biomarkers selected from CD4, CD8, CD56, CD57 and CCR7 and combinations thereof.

3. The method of claim 1, wherein the tuberculosis assessment is a treatment assessment.

4. The method of claim 3, further comprising identifying a subpopulation of the cellular sample having an expression level above a threshold expression level for CD58.

5. The method of claim 1, wherein the tuberculosis assessment is a diagnosis.

6. The method of claim 5, further comprising identifying a subpopulation of the cellular sample having an expression level below a threshold expression level of one or more tuberculosis host biomarkers selected from the group consisting of CD8 and CD57 and combinations thereof.

7. The method of claim 5 or 6, further comprising identifying a subpopulation of the cellular sample having an expression level below a threshold expression level of fMLP r.

8. The method of claim 5 or 6, wherein the diagnosis comprises obtaining a tuberculosis diagnosis for a non-tuberculosis lung disease.

9. The method of claim 1, wherein the tuberculosis assessment comprises a prediction of the likelihood of a positive treatment outcome or a negative treatment outcome.

10. The method of claim 9, further comprising identifying a subpopulation of the cellular sample having an expression level below a threshold expression level of one or more tuberculosis host biomarkers selected from the group consisting of CD18, CD11a, CD50, CD48, CD53, CD62P, CD81, CD45RO, and CD4v4 and combinations thereof.

11. The method of any of claims 1 to 10, wherein the cellular sample is a blood sample.

12. The method of any of claims 1 to 11, wherein the identifying comprises flow cytometry.

13. A kit comprising:
a collection of two or more detectably labeled tuberculosis host biomarker specific binding members, wherein the tuberculosis host biomarkers are selected from the group consisting of CD120b, CD126, CD62L, fMLP r and combinations thereof.

14. A flow cytometry system comprising:
a flow cytometer comprising a flow cell;
a light source configured to direct light to an assay region of the flow cell;
a first detector configured to receive light of a first emission wavelength emitted by a first detectably labeled tuberculosis host biomarker specific binding member present in a cellular sample in the assay region; and
a signal processing module configured to receive signals from the first detector and output a result of whether a subpopulation of cells bound to the first detectably labeled tuberculosis host biomarker specific binding member is present in the cellular sample.

15. A computer readable medium comprising programming for execution by a computer, comprising:
instructions for analyzing signals produced by a detector configured to receive light of a emission wavelength emitted by a detectably labeled tuberculosis host biomarker specific binding member to produce data;
instructions for storing the data on a computer readable medium; and
instructions for outputting the data.
